(19) United States
(12) Patent Application Publication
(10) Pub. No.: US 2018/0156826 A1
(43) Pub. Date: Jun. 7, 2018

(54) COMPOSITIONS AND METHODS FOR DIAGNOSIS OF AUTISM SPECTRUM DISORDER

(71) Applicant: The Board of Regents of the University of Texas System, Austin, TX (US)

(72) Inventors: Dwight GERMAN, Dallas, TX (US); Umar YAZDANI, Plano, TX (US); Sayed ZAMAN, Fort Worth, TX (US); Laura HOLLENBECK, Austin, TX (US)

(73) Assignee: The Board of Regents of the University of Texas Sy stem, Austin, TX (US)

(21) Appl. No.: 15/580,576

(22) PCT Filed: Jun. 3, 2016
(86) PCT No.: PCT/US2016/035640

§ 371 Date: Dec. 7, 2017

Related U.S. Application Data
(60) Provisional application No. 62/243,893, filed on Oct. 20, 2015.
(60) Provisional application No. 62/172,603, filed on Jun. 8, 2015.

(51) Int. Cl. G01N 33/68

(52) U.S. Cl. CPC G01N 33/6896; G01N 2333/59; G01N 2800/30; G01N 2800/28; G01N 2333/5421

(57) ABSTRACT

The present disclosure provides for the identification of biomarkers that are diagnostic for Autism Spectrum Disorder (ASD). The biomarkers include thyroid stimulating hormone (TSH), interleukin 8 (IL-8) and a peptiod recognized by antibodies.

This application claims benefit of priority to U.S. Provisional Application Ser. No. 62/172,603, filed Jun. 8, 2015, and U.S. Provisional Application Ser. No. 62/243,893, filed Oct. 20, 2015, respectively, the entire contents of both applications being hereby incorporated by reference.

This invention was made with government support under Grant Number W81XWH-12-1-520 awarded by the Department of Defense. The government has certain rights in the invention.

### BACKGROUND

### 1. Field

The present disclosure relates generally to the fields of molecular biology, immunology and medicine. More particularly, it concerns the identification of biomarkers for Autism Spectrum Disorder (ASD). The biomarkers include proteins found in blood samples from young males, including hormones, antibodies and cytokines.

### 2. Description of Related Art

Autism spectrum disorder (ASD) is a neurodevelopmental disorder characterized by deficits in social communication and social interaction, and restricted, repetitive patterns of behavior, interests or activities (American Psychiatric Association, 2013). ASD is the fastest growing developmental disability today, affecting more children than cancer, diabetes, and AIDs combined. It affects one out of every 68 children in the U.S., and it is more often found among boys than girls (CDC, 2014). Many genes have been identified that are related to the disorder and even de novo mutations have been found to occur (O'Roak et al., 2014); however, there is great genetic heterogeneity in ASD as recently shown in 85 quartet families where the majority of the siblings with ASD (70%) did not share the same genetic mutation (Yuen et al., 2014). While ASD appears to be on the rise, it is unclear whether the growing number of diagnoses reveals a real increase or comes from improved detection and/or changes to diagnostic criteria.

The immune system has been linked with ASD (Ashwood et al., 2006). Abnormalities in both serum antibody concentrations and T cells have been reported for ASD compared to typically developing (TD) children (Warren et al., 1990; Singh, 2009 and Ashwood et al., 2011). Immunological anomalies in children with ASD include altered cytokine profiles (Jyonouchi et al., 2002; Jyonouchi et al., 2005 and Molloy et al., 2006), decreased immunoglobulin levels (Heuer et al, 2008.), altered cellular immunity (Enstrom et al., 2009) and neuroinflammation (Pardo et al., 2005). Autoimmunity has also been described for autism with several studies reporting circulating autoantibodies to neural antigens (Enstrom et al., 2009 and Wills et al., 2009). This provides yet another avenue of possible exploration in terms of ASD markers.

Current diagnostic methods and screening tools are somewhat subjective and are difficult to assess in younger children, which can often result in missed opportunities for early intervention. A biological marker that could predict ASD risk, assist in early diagnosis or even identify potential therapeutic targets would have great clinical utility (Hewitson, 2013). While biomarker research in ASD has greatly increased in recent years (e.g., Glatt et al., 2012; Momeni et al., 2012; Mizejewski et al., 2013; West et al., 2014; Ngounou et al., 2015), progress has been limited by a number of factors and no universal biological markers for ASD have yet been identified. One of the biggest issues in developing biological markers for ASD is the heterogeneity of the disorder. There is wide variation in symptoms among children with ASD and this is further complicated by a number of co-morbid factors associated with the disorder (Amaral, 2011).

Thus, there remains a need for diagnostic procedures for ASD that are (i) accurate and objective, (ii) simple and reproducible, and (iii) useful in both early and late stage case.

### SUMMARY

The present disclose provides a method of identifying a male subject having or at risk of developing Autism Spectrum Disorder (ASD) comprising (a) providing a blood product sample from a male subject; (b) determining the levels of thyroid stimulating hormone (TSH) and interleukin 8 (IL-8) said sample; and (c) identifying said subject as having or at risk of developing ASD when the level of IL-8 are elevated as compared to levels observed in normal subjects, when the level of TSH are reduced as compared to levels in normal subjects. The method may further comprise determining the level of antibodies binding to a peptoid having the following structure:

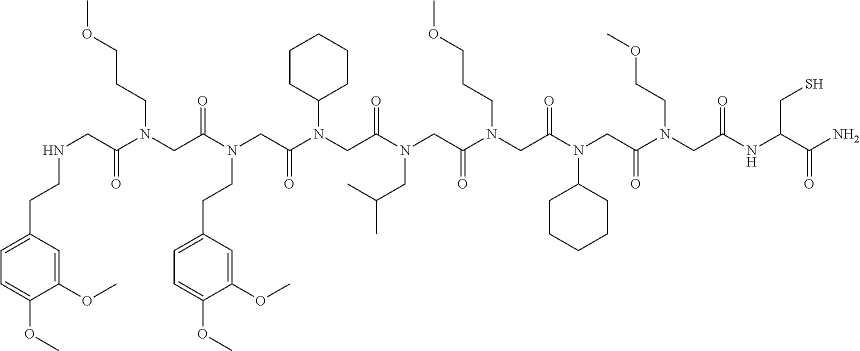 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

and further identifying said subject as having or at risk of developing ASD when the level of said antibodies is reduced as compared to levels observed in normal subjects.

The subject may be suspected as having ASD. The sample may be whole blood or serum. The subject may be age about 12 or younger, about 1-10 years of age, about 2-8 years of age, or about 1 year of age. The peptoid may be located on a solid support, and determining in step (c) may comprise measuring antibodies bound to said solid support. The solid support may be a bead, a chip, a filter, a dipstick, a slide, a membrane, a polymer matrix, a plate or a well. Determining in step (b) may comprise a quantitative immunoassay, such as quantitative ELISA, RIA, FIA, and electrochemiluminescence. The method may further comprise obtaining the sample from the subject, and/or may further comprise examining the level of one or more of ferritin, alpha 1 microglobulin, apolipoprotein E, apolipoprotein H, AXL receptor tyrosine kinase, chromogranin A, monocyte induced by gamma interferon, monocyte chemotactic protein 4, and/or stem cell factor in said sample. The antibodies of step (c) may be IgG1 antibodies.

In another embodiment, there is provided a peptoid having the formula:

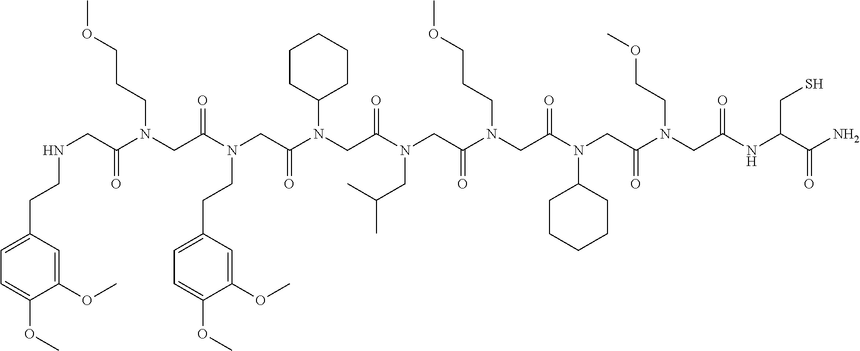 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

Also provided is a solid support having fixed thereto a peptoid having the formula

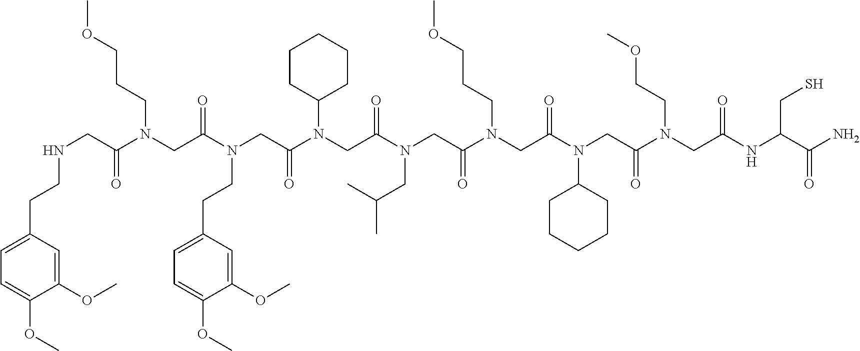 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

The solid support maybe a bead, a chip, a filter, a dipstick, a slide, a membrane, a polymer matrix, a plate or a well. The solid support may further comprise one or more agents for performing a positive-control and/or negative-control reactions for analytes found in a blood sample.

Finally, there is provided a kit comprising a peptoid having the formula

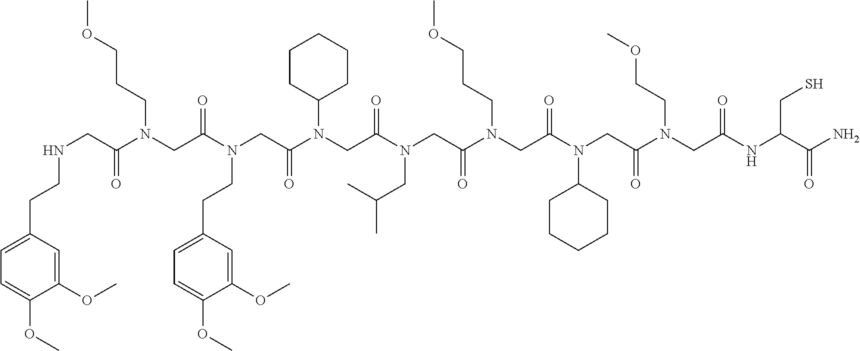 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

The kit may further comprise a support to which said peptoid is afixed. The The kit may further comprising one or more of (a) a reagent for detecting thyroid stimulating hormone levels in a blood product sample; and/or (b) a reagent for detecting interleukin 8 levels in a blood product sample. The kit may also further comprise (d) a reagent or reagents for detecting levels of one or more of ferritin, alpha 1 microglobulin, apolipoprotein E, apolipoprotein H, AXL receptor tyrosine kinase, chromogranin A, monocyte induced by gamma interferon, monocyte chemotactic protein 4, and/or stem cell factor in a blood product sample; (e) a diluent or buffer; and or (f) a container for receiving a blood product sample. The reagent may be an antibody. The kit may also further comprise one or more agents for performing a positive-control and/or negative-control reactions for analytes found in a blood sample.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the disclosure, and vice versa. Furthermore, compositions and kits of the disclosure can be used to achieve methods of the disclosure.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Serum protein measurements on the MSD platform. (FIG. 1A) TSH levels are significantly lower in ASD boys. (FIG. 1B) IL-8 levels are significantly higher in the ASD boys. *p<0.03, **p<0.01.

FIG. 2. Configuration of the first library used to screen for ASD-related compounds. Abbreviations: Met=methionine; Nall=allylamine; Nasp=glycine; Ncha=cyclohexylamine; Nffa=furfurylamine; Nleu=isobutylamine; Nmba=(R)-methylbenzylamine; Nmea=2-methoxyethylamine; Nmpa=3-methoxypropylamine; Nphe=benzylamine; Npip=piperonylamine; Npyr=N-(3′-aminopropyl)-2-pyrrolidinone; Nser=ethanolamine.

FIG. 3. On-bead magnetic screening. A one-bead one-compound (OBOC) library of thousands of unique peptoid compounds bound to TentaGel beads is incubated with control serum, here serum pooled from TD subjects. The library is then incubated with anti-human IgG-labeled magnetic nanoparticles so that beads having bound IgG from the serum can be sorted out using a strong magnet. The library is initially depleted of beads that bind IgG from the control serum, and then incubated with target serum, here serum pooled from ASD subjects. After incubation with the magnetic nanoparticles again, the newly magnetized beads, called “hits”, are isolated. Peptoid compounds are cleaved from each of the “hit” beads and their sequences are assessed by MS/MS. These “hit” compounds are then re-synthesized and validated on ELISA plates for their ability to detect target IgG.

FIGS. 4A-D. Serum IgG binding to the ASD1 peptoid. (FIG. 4A) Titration of IgG binding to ASD1 using serum pooled from 10 TD males (TD-M) and 10 ASD males (ASD-M) demonstrates ASD1's ability to differentiate between the two groups. (FIG. 4B) Detecting IgG1 subclass instead of total IgG amplifies this differentiation. (FIG. 4C) IgG1 binding of individual ASD (n=50) and TD (n=43) male serum samples (1:100 dilution) to ASD1 significantly differs with TD>ASD. In addition, IgG1 binding of older adult male (AM) serum samples (n=53) to ASD1 is significantly lower than TD males, and not different from ASD males. Results are shown here as ratios of each sample's absorbance to that of a control pool included on the plate. Error bars show SEM. (FIG. 4D) Receiver-operating characteristic curve for ASD1's ability to discriminate between ASD and TD males.

FIG. 5. Assessment of proteins that bind to ASD1. ASD1 peptoid was immobilized and incubated with pooled serum from ASD or TD males. Serum was removed and what proteins were left bound to ASD1 were eluted out and evaluated by gel electrophoresis and Coommassie Blue staining. Lane 1 shows ASD1 pull-down analytes from the ASD serum pool and Lane 2 shows the pull-down from the TD serum pool. Both show a single band at ˜55-60 kD that is higher in intensity for the TD male analyte.

FIG. 6. Serum protein measurements from the RBM Luminex platform. A panel of 11 serum proteins were combined to predict ASD among ASD and TD boys (n=28/group). Using random forest analysis, the importance for each protein in predicting ASD vs. TD is illustrated.

FIG. 7. Descriptive statistics for TSH and IL8 levels in ASD and TD boys using samples run from the same subjects.

FIG. 8. Predicting ASD from TSH and IL8 together.

FIGS. 9A-D. Serum protein measurements on the MSD platform. (FIG. 9A) TSH levels are significantly lower in ASD boys (p=0.007), (FIG. 9B) ROC curve for TSH area=0.674, (FIG. 9C) IL-8 levels are significantly higher in ASD boys (p=0.025), and (FIG. 9D) ROC curve for IL8 area=0.654. Mann Whitney U-tests.

### DESCRIPTION OF ILLUSTRATIVE EMBODIMENTS

The inventors here describe a study using a panel of over 100 proteins to search for potential biomarkers for autism spectrum disorder (ASD), using serum from ASD boys and typically developing (TD) boys (n=30/group). The tests were conducted in a gender-specific manner since the disorder is approximately four times more common in males (Schaafsma & Pfaff, 2014). Eleven proteins were found to differ between the two groups, and Random Forest analysis indicated that the panel of proteins together could predict ASD with modest accuracy. Three proteins from the panel were further tested on the Meso Scale Discovery (MSD) electrochemiluminescent platform (n=34-41/group): thyroid stimulating hormone (TSH) and interleukin 8 (IL-8) and monokine induced by gamma interferon (MIG). The inventors found significantly lower TSH levels and higher IL-8 levels in the ASD boys using both platforms. The diagnostic accuracy for predicting ASD based upon the TSH level was 66.6% and for IL-8 it was 65.2%; however, using both proteins together, the diagnostic accuracy increased to 84.2%. These data indicate that a panel of serum proteins can be useful as a biomarker for ASD in boys.

The inventors further screened highly complex libraries of peptoids (oligo-N-substituted glycines) were screened for compounds that preferentially bind IgG from subjects with ASD over that of typically developing (TD) subjects. Unexpectedly, many peptoids were identified that preferentially bound IgG from TD males. One of these peptoids was studied further and found to bind significantly higher levels of the IgG1 subtype in serum from TD boys (n=43) compared to ASD boys (n=50), as well as compared to older adult males (n=53). Together these data suggest that ASD boys have reduced levels of an IgG1 antibody, which resembles the level found normally with advanced age. This peptoid was 66% accurate in predicting ASD.

By combining the protein biomarkers discussed above, with the antibody binding to the identified peptoid, the inventors provide a robust, accurate and sensitive assay for predicting/diagnosing ASD. These and other aspects of the disclosure are described in detail below.

### I. AUTISM SPECTRUM DISORDER

The present disclosure, as discussed above, provides for the diagnosis of autism spectrum disorder (ASD), which describes a range of conditions classified as neurodevelopmental disorders in the fifth revision of the American Psychiatric Association's Diagnostic and Statistical Manual of Mental Disorders 5th edition (DSM-5). The DSM-5, published in 2013, redefined the autism spectrum to encompass the previous (DSM-IV-TR) diagnoses of autism, Asperger syndrome, pervasive developmental disorder not otherwise specified (PDD-NOS), and childhood disintegrative disorder. These disorders are characterized by social deficits and communication difficulties, stereotyped or repetitive behaviors and interests, sensory issues, and in some cases, cognitive delays.

A revision to ASD was proposed in the Diagnostic and Statistical Manual of Mental Disorders version 5 (DSM-5), released May 2013. The new diagnosis encompasses previous diagnoses of autistic disorder, Asperger's disorder, childhood disintegrative disorder, and PDD-NOS. Rather than categorizing these diagnoses, the DSM-5 will adopt a dimensional approach to diagnosing disorders that fall underneath the autism spectrum umbrella. It is thought that individuals with ASDs are best represented as a single diagnostic category because they demonstrate similar types of symptoms and are better differentiated by clinical specifiers (i.e., dimensions of severity) and associated features (i.e., known genetic disorders, epilepsy and intellectual disability). An additional change to the DSM includes collapsing social and communication deficits into one domain. Thus, an individual with an ASD diagnosis will be described in terms of severity of social communication symptoms, severity of fixated or restricted behaviors or interests and associated features. The restriction of onset age has also been loosened from 3 years of age to “early developmental period,” with a note that symptoms may manifest later when demands exceed capabilities.

Autism forms the core of the autism spectrum disorders. Asperger syndrome is closest to autism in signs and likely causes; unlike autism, people with Asperger syndrome have no significant delay in language development. PDD-NOS is diagnosed when the criteria are not met for a more specific disorder. Some sources also include Rett syndrome and childhood disintegrative disorder, which share several signs with autism but may have unrelated causes; other sources differentiate them from ASD, but group all of the above conditions into the pervasive developmental disorders.

Autism, Asperger syndrome, and PDD-NOS are sometimes called the autistic disorders instead of ASD, whereas autism itself is often called autistic disorder, childhood autism, or infantile autism. Although the older term pervasive developmental disorder and the newer term autism spectrum disorder largely or entirely overlap, the former was intended to describe a specific set of diagnostic labels, whereas the latter refers to a postulated spectrum disorder linking various conditions. ASD, in turn, is a subset of the broader autism phenotype (BAP), which describes individuals who may not have ASD but do have autistic-like traits, such as avoiding eye contact.

Under the DSM-5, autism is characterized by persistent deficits in social communication and interaction across multiple contexts, as well as restricted, repetitive patterns of behavior, interests, or activities. These deficits are present in early childhood, and lead to clinically significant functional impairment. There is also a unique form of autism called autistic savantism, where a child can display outstanding skills in music, art, and numbers with no practice.

Asperger syndrome was distinguished from autism in the DSM-IV by the lack of delay or deviance in early language development. Additionally, individuals diagnosed with Asperger syndrome did not have significant cognitive delays. PDD-NOS was considered “subthreshold autism” and “atypical autism” because it was often characterized by milder symptoms of autism or symptoms in only one domain (such as social difficulties). In the DSM-5, both of these diagnoses have been subsumed into autism spectrum disorder.

A. Developmental Course

Although autism spectrum disorders are thought to follow two possible developmental courses, most parents report that symptom onset occurred within the first year of life. One course of development follows a gradual course of onset in which parents report concerns in development over the first two years of life and diagnosis is made around 3-4 years of age. Some of the early signs of ASDs in this course include decreased looking at faces, failure to turn when name is called, failure to show interests by showing or pointing, and delayed pretend play. A second course of development is characterized by normal or near-normal development followed by loss of skills or regression in the first 2-3 years. Regression may occur in a variety of domains, including communication, social, cognitive, and self-help skills; however, the most common regression is loss of language. There continues to be a debate over the differential outcomes based on these two developmental courses. Some studies suggest that regression is associated with poorer outcomes and others report no differences between those with early gradual onset and those who experience a regression period. Overall, the prognosis is poor for persons with classical (Kanner-type) autism with respect to academic achievement and poor to below-average for persons across the autism spectrum with respect to independent living abilities; in each case, a lack of early intervention exacerbates the odds against success. However, many individuals show improvements as they grow older. The two best predictors of favorable outcome in autism are the absence of intellectual disability and the development of some communicative speech prior to five years of age. Overall, the literature stresses the importance of early intervention in achieving positive longitudinal outcomes.

While a specific cause or specific causes of autism spectrum disorders has yet to be found, many risk factors have been identified in the research literature that may contribute to the development of an ASD. These risk factors include genetics, prenatal and perinatal factors, neuroanatomical abnormalities, and environmental factors. It is possible to identify general risk factors, but much more difficult to pinpoint specific factors. In the current state of knowledge, prediction can only be of a global nature and therefore requires the use of general markers.

B. Genetic Risk Factors

The results of family and twin studies suggest that genetic factors play a role in the etiology of autism and other pervasive developmental disorders. Studies have consistently found that the prevalence of autism in siblings of autistic children is approximately 15 to 30 times greater than the rate in the general population. In addition, research suggests that there is a much higher concordance rate among monozygotic twins compared to dizygotic twins. It appears that there is no single gene that can account for autism. Instead, there seem to be multiple genes involved, each of which is a risk factor for part of the autism syndrome through various groups.

C. Diagnosis

Evidence-Based Assessment.

ASD can be detected as early as eighteen months or even younger in some cases. A reliable diagnosis can usually be made by the age of two. The diverse expressions of ASD symptoms pose diagnostic challenges to clinicians. Individuals with an ASD may present at various times of development (e.g., toddler, child, or adolescent) and symptom expression may vary over the course of development. Furthermore, clinicians are required to differentiate among the different pervasive developmental disorders as well as other disorders such as intellectual disability not associated with a pervasive developmental disorder, specific developmental disorders (e.g., language), and early onset schizophrenia, as well as the social-cognitive deficits caused by brain damage from alcohol abuse.

Considering the unique challenges associated with diagnosing ASD, specific practice parameters for the assessment of ASD have been published by the American Academy of Neurology, the American Academy of Child and Adolescent Psychiatry, and a consensus panel with representation from various professional societies. The practice parameters outlined by these societies include an initial screening of children by general practitioners (i.e., “Level 1 screening”) and for children who fail the initial screening, a comprehensive diagnostic assessment by experienced clinicians (i.e., “Level 2 evaluation”). Furthermore, it has been suggested that assessments of children with suspected ASD be evaluated within a developmental framework, include multiple informants (e.g., parents and teachers) from diverse contexts (e.g., home and school), and employ a multidisciplinary team of professionals (e.g., clinical psychologists, neuropsychologists, and psychiatrists).

After a child fails an initial screening, psychologists administer various psychological assessment tools to assess for ASD. Amongst these measurements, the Autism Diagnostic Interview-Revised (ADI-R) and the Autism Diagnostic Observation Schedule (ADOS) are considered the “gold standards” for assessing autistic children. The ADI-R is a semi-structured parent interview that probes for symptoms of autism by evaluating a child's current behavior and developmental history. The ADOS is a semistructured interactive evaluation of ASD symptoms that is used to measure social and communication abilities by eliciting a number of opportunities (or “presses”) for spontaneous behaviors (e.g., eye contact) in standardized context. Various other questionnaires (e.g., The Childhood Autism Rating Scale) and tests of cognitive functioning (e.g., The Peabody Picture Vocabulary Test) are typically included in an ASD assessment battery.

D. Comorbidity

Autism spectrum disorders tend to be highly comorbid with other disorders. Comorbidity may increase with age and may worsen the course of youth with ASDs and make intervention/treatment more difficult. Distinguishing between ASDs and other diagnoses can be challenging because the traits of ASDs often overlap with symptoms of other disorders and the characteristics of ASDs make traditional diagnostic procedures difficult.

The most common medical condition occurring in individuals with autism spectrum disorders is seizure disorder or epilepsy, which occurs in 11-39% of individuals with ASD. Tuberous sclerosis, a medical condition in which non-malignant tumors grow in the brain and on other vital organs, occurs in 1-4% of individuals with ASDs.

Intellectual disabilities are some of the most common comorbid disorders with ASDs. Recent estimates suggest that 40-69% of individuals with ASD have some degree of mental retardation, with females more likely to be in severe range of mental retardation. Learning disabilities are also highly comorbid in individuals with an ASD. Approximately 25-75% of individuals with an ASD also have some degree of learning disability.

A variety of anxiety disorders tend to co-occur with autism spectrum disorders, with overall comorbidity rates of 7-84%. Rates of comorbid depression in individuals with an ASD range from 4-58%.

Deficits in ASD are often linked to behavior problems, such as difficulties following directions, being cooperative, and doing things on other people's terms. Symptoms similar to those of Attention Deficit Hyperactivity Disorder (ADHD) can be part of an ASD diagnosis. Sensory processing disorder is also comorbid with ASD, with comorbidity rates of 42-88%.

E. Management

There is no known cure for autism. The main goals of treatment are to lessen associated deficits and family distress, and to increase quality of life and functional independence. No single treatment is best and treatment is typically tailored to the child's needs. Intensive, sustained special education programs and behavior therapy early in life can help children acquire self-care, social, and job skills. Available approaches include applied behavior analysis, developmental models, structured teaching, speech and language therapy, social skills therapy, and occupational therapy. There has been increasing attention to the development of evidenced-based interventions for young children with ASDs. Unresearched alternative therapies have also been implemented (for example, vitamin therapy and acupuncture). Although evidenced-based interventions for autistic children vary in their methods, many adopt a psychoeducational approach to enhancing cognitive, communication and social skills while minimizing problem behaviors. It has been argued that no single treatment is best and treatment is typically tailored to the child's needs.

One of the most empirically supported intervention approaches is applied behavioral analysis, particularly in regard to early intensive home-based therapy. Although ABA therapy has a strong research base, other studies have found that this approach may be limited by diagnostic severity and IQ.

### II. DIAGNOSTIC DETERMINATIONS IN ASD

The present disclosure, in one aspect, can provide a diagnosis for ASD. This will permit doctors to more readily discern between ASD and other diseases with overlapping sets of symptoms, and thus having correctly identified the underlying physiologic basis for a patient's symptoms, open up early intervention and disease management. Indeed, because treatments for ASD do not prevent or cure disease, the ability to provide an early diagnosis for these diseases is critical to delaying the onset of more severe symptoms. In addition, being able to provide patients with the correct drugs to address their symptoms without “trial and error” that sometimes results from incorrect diagnosis, will significantly reduce the cost of care, and avoid patient discomfort and possible harm.

These assays will generally use a blood sample, such as whole blood, serum or plasma. However, other samples such as tear, saliva, sputum, cerebrospinal fluid, semen or urine may prove useful as well.

In assessing the biomarker levels in the subject, the observed reactivity patterns can be compared to a standard. The standard may rely on known levels for both diseased and normal subjects, and may therefore obviate the need for a the user to provide anything but a reaction control, i.e., a control showing that the reagents and conditions necessary for a positive reaction are present. Alternatively, one may choose to run an actual control which comprises a similar sample from an actual person of known healthy or diseased status. In addition, one may run a series of samples from the same subject over time looking for a trend of increasing/decreasing biomarker levels as an indication of disease progression.

There are a number of different ways to detect biomarkers according to the present disclosure. One type of assay will involve, or be modeled upon, antibody-based assays, including formats such as enzyme linked immunosorbent assays (ELISAs), radioimmunoassays (RIAs), immunoradiometric assays, fluoroimmunoassays, chemiluminescent assays, bioluminescent assays, FACS, FRET and Western blot to mention a few. The steps of various immunodetection methods have been described in the scientific literature, such as, e.g., Doolittle and Ben-Zeev (1999), Gulbis and Galand (1993), De Jager et al. (1993), and Nakamura et al. (1987). In general, such assays will involve the use of a capture agent (peptoid or antibody) disposed on a support.

The solid support may be in the form of a column matrix, bead, filter, membrane, stick, plate, or well and the sample will be applied to the immobilized peptoid. After contacting with the sample, unwanted (non-specifically bound) components will be washed from the support, biomarkers complexed with the capture agent, which are then detected using various means, such as subsequent addition of antibodies that recognize the biomarkers or the capture agent (which are not available if bound to a biomarker).

Contacting the chosen biological sample under effective conditions and for a period of time sufficient to allow the formation of biomarker-capture agent complexes is generally a matter of simply contacting the sample with the capture agent and incubating the mixture for a period of time long enough for the biomarkers to bind the capture agent. After this time, the resulting sample, such as a plate, filter or blot, will generally be washed to remove any non-specifically bound cell species or debris, allowing only those biomarkers specifically bound to the immobilized capture agent to be detected.

In general, the detection of biological complex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

Various other formats are contemplated and are well known to those of skill in the art. Discussed below are three particular assays envisioned to have ready applicability to the present disclosure.

A. Immunassays

Immunoassays, in their most simple and direct sense, are binding assays. Certain immunoassays finding particular use in the present disclosure are various types of enzyme linked immunosorbent assays (ELISAs) and radioimmunoassays (RIA) known in the art.

In ELISAs, the binding ligand may be immobilized onto a selected surface, such as a well in a polystyrene microtiter plate. Then, a test composition suspected of containing the target is added to the wells. After binding and washing to remove non-specifically bound complexes, the bound target may be detected. Detection may be achieved by the addition of another binding ligand linked to a detectable label. This type of assay is analogous to a simple “sandwich ELISA” where the binding of the labeled agent is directed at portion of the target not bound by the ligand fixed to the support. Detection may also be achieved by the addition of a labeled molecule that binds to the support-bound ligand, and the absence of a signal shows competition for the support bound ligand by the target in the sample. Optionally, if the target is an antibody, the detection can be achieved by the addition of a labeled second antibody that has binding affinity for the first antibody (Fc).

In another exemplary ELISA, the samples suspected of containing the targets are immobilized onto a well surface and then contacted with labeled peptoids or antibodies of the present disclosure. After binding and washing to remove non-specifically bound immune complexes, the bound labeled peptoids and antibodies are detected.

Irrespective of the format employed, ELISAs have certain features in common, such as coating, incubating and binding, washing to remove non-specifically bound species, and detecting the bound immune complexes. Because of the simple and predictable chemistry of the peptoids, they can be attached to the support by means of a specific chemical reaction.

“Under conditions effective to allow immune complex formation” means that the conditions preferably include diluting the sample with solutions such as BSA, bovine y globulin (BGG) or phosphate buffered saline (PBS)/Tween. These added agents also tend to assist in the reduction of non-specific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

Detection may utilize an enzyme that will generate color development upon incubating with an appropriate chromogenic substrate. Thus, for example, one will desire to contact or incubate the immune complex with a urease, glucose oxidase, alkaline phosphatase or hydrogen peroxidase-conjugated antibody or peptoid for a period of time and under conditions that favor the development of that immune complex (e.g., incubation for 2 hours at room temperature in a PBS-containing solution such as PBS-Tween). Obviously, other formats (RIA, FIA) will use other types of labels in an analogous fashion.

After incubation with the labeled antibody or peptoid, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

A particular type of label/read out for an immunoassay contemplated here is electrochemiluminescence or electrogenerated chemiluminescence (ECL), a kind of luminescence produced during electrochemical reactions in solutions. In electrogenerated chemiluminescence, electrochemically generated intermediates undergo a highly exergonic reaction to produce an electronically excited state that then emits light upon relaxation to a lower-level state. This wavelength of the emitted photon of light corresponds to the energy gap between these two states. ECL excitation can be caused by energetic electron transfer (redox) reactions of electrogenerated species. Such luminescence excitation is a form of chemiluminescence where one/all reactants are produced electrochemically on the electrodes.

ECL is usually observed during application of potential (several volts) to electrodes of electrochemical cell that contains solution of luminescent species (polycyclic aromatic hydrocarbons, metal complexes, Quantum Dots or Nanoparticles) in aprotic organic solvent (ECL composition). In organic solvents, both oxidized and reduced forms of luminescent species can be produced at different electrodes simultaneously or at a single one by sweeping its potential between oxidation and reduction. The excitation energy is obtained from recombination of oxidized and reduced species.

In aqueous medium, which is mostly used for analytical applications, simultaneous oxidation and reduction of luminescent species is difficult to achieve due to electrochemical splitting of water itself so the ECL reaction with the coreactants is used. In the later case luminescent species are oxidized at the electrode together with the coreactant which gives a strong reducing agent after some chemical transformations (the oxidative reduction mechanism).

ECL proved to be very useful in analytical applications as a highly sensitive and selective method. It combines analytical advantages of chemiluminescent analysis (absence of background optical signal) with ease of reaction control by applying electrode potential. As an analytical technique it presents outstanding advantages over other common analytical methods due to its versatility, simplified optical setup compared with photoluminescence (PL), and good temporal and spatial control compared with chemiluminescence (CL). Enhanced selectivity of ECL analysis is reached by variation of electrode potential thus controlling species that are oxidized/reduced at the electrode and take part in ECL reaction (see electrochemical analysis).

It generally uses Ruthenium complexes, especially [Ru (Bpy)3]²⁺ (which releases a photon at ˜620 nm) regenerating with TPA (Tripropylamine) in liquid phase or liquid-solid interface. It can be used as monolayer immobilized on an electrode surface (made, e.g., of nafion, or special thin films made by Langmuir-Blogett technique or self-assembly technique) or as a coreactant or more commonly as a tag and used in HPLC, Ru tagged antibody based immunoassays, Ru Tagged DNA probes for PCR, etc., NADH or H₂O₂ generation based biosensors, oxalate and organic amine detection and many other applications and can be detected from picomolar sensitivity to dynamic range of more than six orders of magnitude. Photon detection is done with photomultiplier tubes (PMT) or silicon photodiode or gold coated fiber-optic sensors. The importance of ECL techniques detection for bio-related applications has been well established. ECL is heavily used commercially for many clinical lab applications.

B. Peptoids

In one embodiment, there is provided a peptoid that is recognized by antibodies in serum. Peptoids, or poly-N-substituted glycines, are a class of peptidomimetics whose side chains are appended to the nitrogen atom of the peptide backbone, rather than to the α-carbons (as they are in amino acids). In peptoids, the side chain is connected to the nitrogen of the peptide backbone, instead of the α-carbon as in peptides. Notably, peptoids lack the amide hydrogen which is responsible for many of the Secondary structure elements in peptides and proteins.

Following the sub-monomer protocol originally created by Ron Zuckermann, each residue is installed in two steps: acylation and displacement. In the acylation step a haloacetic acid, typically bromoacetic acid activated by diisopropylcarbodiimide reacts with the amine of the previous residue. In the displacement step (a classical S_{N}2 reaction), an amine displaces the halide to form the N-substituted glycine residue. The submonomer approach allows the use of any commercially available or synthetically accessible amine with great potential for Combinatorial chemistry. Like D-Peptides and β peptides, peptoids are completely resistant to proteolysis, and are therefore advantageous for therapeutic applications where proteolysis is a major issue. Since secondary structure in peptoids does not involve hydrogen bonding, it is not typically denatured by solvent, temperature, or chemical denaturants such as urea.

Notably, since the amino portion of the amino acid results from the use of any amine, thousands of commercially available amines can be used to generate unprecedented chemical diversity at each position at costs far lower than would be required for similar peptides or peptidomimetics. To date, at least 230 different amines have been used as side chains in peptoids.

Peptoid oligomers are known to be conformationally unstable, due to the flexibility of the main-chain methylene groups and the absence of stabilizing hydrogen bond interactions along the backbone. Nevertheless, through the choice of appropriate side chains, it is possible to form specific steric or electronic interactions that favor the formation of stable secondary structures like helices, especially peptoids with C-α-branched side chains are known to adopt structure analogous to polyproline I helix. Different strategies have been employed to predict and characterize peptoid secondary structure, with the ultimate goal of developing fully folded peptoid protein structures. The cis/trans amide bond isomerization still leads to a conformational heterogeneity which does not allow for the formation of homogeneous peptoid foldamers. Nonetheless scientists were able to find trans-inducer N-Aryl side chains promoting polyproline type II helix, and strong cis-inducer such as bulky naphtylethyl and tert-butyl side chains. It was also found that n→π* interactions can modulate the ratio of cis/trans amide bond conformers, until reaching a complete control of the cis conformer in the peptoid backbone using a functionalizable triazolium side chain.

Many researchers use peptoids as part of a large array or library to probe the binding diversity of samples, for example, those containing antibodies. These libraries are generated using randomized addition of peptoid monomers to create a library with N^{X} diversity, with N being the number of different monomers, and X being the number of residues in the peptoid. A particular approach is the “one-bead one-compound” method, which the peptoids are typically generated on a bead surface. In certain aspects, tentagel beads or resin can be used. One of the most common microsphere formations is tentagel, a styrene-polyethylene glycol co-polymer. These microspheres are unswollen in nonpolar solvents such as hexane and swell approximately 20-40% in volume upon exposure to a more polar or aqueous media. Peptoids can be synthesized by sequential conjugation of each residue added to the peptoid, using peptoid synthesis chemistry. The split synthesis method yields beads each of which comprises multiple copies of a single peptoid sequence per bead.

C. Detection Kits

In still further embodiments, the present disclosure concerns detection kits for use with the methods described above. Peptoids according to the present disclosure, along with antibodies, will be included in the kit. The kits will thus comprise, in suitable container means, one or more peptoids that bind antibodies in ASD blood/serum, optionally linked to a detection reagent and/or a support.

In certain embodiments where the peptoid and/or antibody is pre-bound to a solid support, the support is provide and includes a column matrix, bead, stick or well of a microtiter plate. The immunodetection reagents of the kit may take any one of a variety of forms, including those detectable labels that are associated with or linked to the given peptoid or antibody. Exemplary antibodies are those having binding affinity for the TSH, IL-8 or other targets shown in Table 1.

The container means of the kits will generally include at least one vial, test tube, flask, bottle, syringe or other container means, into which the peptoid or antibody may be placed, or preferably, suitably aliquoted. The kits of the present disclosure will also typically include a means for containing the peptoid, antibody, and any other reagent containers in close confinement for commercial sale. Such containers may include injection or blow-molded plastic containers into which the desired vials are retained.

In addition, the kits may contain positive or negative control antibodies or antigens. They may also contain tools/reagents for obtaining and/or processing samples, such as blood, blood products, urine, saliva, cerebrospinal fluid, and lymph. Finally, the kits may include instructions for use and interpretation of results.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Methods

Ethics.

The study protocol and all subsequent amendments were submitted by The Johnson Center for Child Health and Development (Austin, Tex.) and approved by the Austin Multi-Institutional Review Board (AMIRB) for ASD and TD subjects.

Study Subjects.

The ASD group was comprised of 41 male subjects age 2.4 to 8.1 years (mean 5.2±1.6 SD) and 10 female subjects age 2.8 to 6.3 years (4.7±1.4). The TD group was comprised of 37 males age 2.0 to 8.7 years (5.1±1.6), and 10 females age 2.2 to 6.7 years (5.1±1.3). Subjects were either recruited directly from The Johnson Center clinic, or through the use of informational study flyers circulated around Austin, Tex. Written informed consent was received from the parent or guardian of all subjects prior to enrollment. For the ASD group, all subjects were assessed by The Johnson Center staff psychologist using both the Autism Diagnostic Observation Schedule (ADOS) and the Autism Diagnostic Interview-Revised (ADI-R). Clinical diagnosis was made based on these data and overall clinical impression using DSM-IV criteria. For this particular study, subjects with a diagnosis of Asperger's Syndrome or Pervasive Developmental Disorder—Not Otherwise Specified, were excluded. For the TD group, all subjects underwent a developmental screening using the Adaptive Behavior Assessment System-Second Edition (ABAS-II) that was assessed by the psychologist. TD subjects were excluded if their score on the ABAS-II suggested possible abnormal development, and the need for further evaluation. TD subjects were also excluded if they had a first- or second-degree relative diagnosed with ASD. Any subjects diagnosed with a genetic, metabolic, or other concurrent physical, mental, or neurological disorder were excluded, as were subjects that were currently taking psychiatric medications (or had taken psychiatric medications within the last 3 months prior to enrollment).

Blood Collection/Storage.

A fasting blood draw was performed on healthy children between the hours of 8-10 a.m. Blood was collected into a 3.5 ml Serum Separation Tube (SST; Vacutainer System; Becton-Dickinson) by a nurse using standard venipuncture technique. The blood was gently mixed in the SST by 5 inversions and then stored upright for clotting at room temperature for 10-15 mins. Blood was then spun immediately after the clotting time in a swing bucket rotor for 15 minutes at 1100-1300 g at room temperature. Serum was removed immediately after centrifugation and transferred into coded cryovials in 0.5 ml aliquots. Aliquots of serum were immediately placed upright in a specimen storage box in a −20° C. freezer for up to 6 hours. Samples were then transferred to a −80° C. freezer for long-term storage.

Analyte Measurements on Rules Based Medicine Platform.

Sample aliquots were coded to remove the possibility of bias and shipped on dry ice to Myriad-Rules Based Medicine (RBM; Austin Tex.) for evaluation using DiscoveryMAP 175+ for quantitative immunoassay of inflammatory molecules and hormones. A total of 30 ASD and 30 TD male serum samples were analyzed. A multianalyte Luminex profiling platform is used containing over 175 protein analytes. Final data were reported as the absolute concentrations in the serum. Some coded duplicate samples were run and used to analyze analyte measurement performance. Those analyte measurements that showed >15% variance were excluded from the data analysis.

Validation Measurements on the Meso Scale Discovery Platform.

Compared with the traditional ELISA approach, the MSD platform shows greater sensitivity and is able to reliably detect different proteins across a broad dynamic range of concentrations (Burguillos, 2013). The assay is based upon electrochemiluminescence technology by using specific capture antibodies coated at corresponding spots on an electric wired microplate. This platform was used to measure TSH, MIG and IL-8 identified as showing the greatest difference in the ASD and TD samples run on the RBM platform. Samples were run in duplicate accordingly to the manufacturer's protocol. Any duplicate value with >15% variance was removed from the final data analysis, and every plate was run with a standard concentration curve.

Statistical Analyses.

The RBM data were analyzed using Random Forest methods. Random forest analysis was developed as an ensemble learning method that utilizes a classification tree as the base classifier (Brieman, 2001). Hundreds of Training and Test sets, of 15 subjects/group, were run to determine the importance of a panel of analytes to correctly identifying ASD subjects. For the MSD data, differences between the ASD and TD groups were analyzed with Mann Whitney U-tests. For comparing the accuracy of two analytes for predicting ASD vs. TD, the inventors used cutscores and area under the curve analyses. Diagnostic accuracy was computed by ROC (Receiver Operation Characteristic) curves using R package; AUC (area under the curve) was calculated using R package DiagnosisMed (V 0.2.2.2), and the optimum probability cutoffs were determined by the software to maximize the accuracy area. The p<0.05 level was considered to be statistically significant.

### Results

Proteins Measured on the RBM Platform.

A total of 184 analytes were measured on the RBM luminex platform, but 51 were undetectable, and 23 exhibited >15% spot-to-spot variance and were therefore omitted from analysis. Eleven of the remaining 110 serum proteins measured were selected using the Random Forest analysis, and 6 of the analytes were significantly different at the p≤0.05 level (n=30 males/group) (see Table 1). The Test sets ability to accurately predict ASD vs. TD most often exhibited areas under the ROC curve=0.60.

TABLE 1
SERUM PROTEINS MEASUREMENTS ON THE RBM PLATFORM

| | Protein names | Change | t-test | Importance |
|---|---|---|---|---|
| 1 | Alpha 1 Microglobulin- | 9%↑ | 0.017359 | 2.309376 |
| | A1Micro | | | |
| 2 | Apolipoprotein E-ApoE | 22%↑ | 0.035705 | −1.86584 |
| 3 | Apolipoprotein H-ApoH | 15%↑ | 0.103483 | 4.153229 |
| 4 | AXL Receptor Tyrosine | 11%↑ | 0.059623 | −0.23316 |
| | Kinase-AXL | | | |
| S | Chromogranin A-CgA | 21%↑ | 0.05041 | −0.41879 |
| 6 | Ferritin-FRTN | 29%↑ | 0.056172 | 3.295529 |
| 7 | Interleukin 8-IL-8 | 31%↑ | 0.043234 | 2.568925 |
| 8 | Monocyte Chemotactic | 18%↑ | 0.06425 | 3.393397 |
| | Protein 4-MCP4 | | | |
| 9 | Monokine Induced by | 26%↑ | 0.166328 | 2.088083 |
| | Gamma Interferon-MIG | | | |
| 10 | Stem Cell Factor-SCF | 16%↑ | 0.008027 | 4.356693 |
| 11 | Thyroid Stimulating | 31%↓ | 0.003567 | 14.63983 |
| | Hormone-TSH | | | |

Proteins Measured on the MSD Platform.

The inventors chose to measure on the MSD platform levels of three of the proteins identified on the RBM platform that showed the greatest percent difference between the ASD and TD groups—TSH, MIG and IL-8. All of the samples run on the RBM platform were also run on the MSD platform, plus in some cases additional samples were included to increase the sample size.

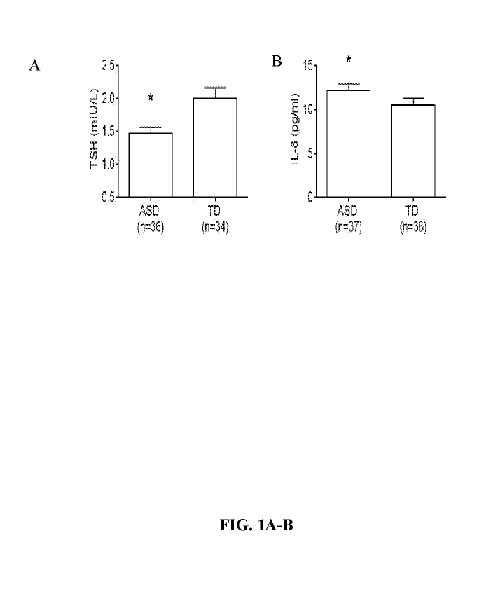

TSH levels were 26% lower in ASD boys (n=36) vs. TD boys (n=34)—1.47±0.09 (mean±SEM) and 2.01±0.16 mlU/l, respectively (p<0.016; Mann Whitney U test) (see FIG. 1A). The area under the ROC curve is 0.666, p=0.016. In ASD and TD females (n=10/group) there was no difference in TSH levels −1.74±0.3 and 1.87±0.3 mlU/L, respectively.

IL-8 levels were 16% higher in ASD boys (n=37) vs. TD boys (n=38)—12.24±0.74 (mean±SEM) and 10.47±0.77 pg/ml, respectively (p<0.015; Mann Whitney U test) (see FIG. 1C). The area under the ROC curve is 0.652, p=0.023.

TABLE 2
THE PERCENT CHANGE IN THE 2 MARKERS
FOR THE ASD MALE SAMPLES ESTIMATED
ON BOTH THE RBM AND MSD PLATFORMS

| | RBM (ASD % change) | MSD (ASD % change) |
|---|---|---|
| TSH (mIU/L) | 31%↓ ** | 26%↓ ** |
| IL-8 (pg/ml) | 31%↑ * | 16↑ ** |

* = p < 0.05;
** p < 0.016-TD vs. ASD

In order to determine whether the accuracy in predicting ASD vs. TD is enhanced by an analysis using a combination of analytes, the inventors selected two analytes to study (TSH and IL-8). Each of the two analytes had an accuracy of 74-76%, but the combination of the two analytes gave an accuracy of 82%. ASD cases were predicted as having TSH levels below 1.86 and IL-8 levels above 10.3. The area under the curve for this model was 0.842 (95% CI: 0.711-0.972; p<0.001).

### Discussion

Using a quantitative immunoassay of inflammatory molecules and hormones, 11 proteins were found to be present in significantly different levels in serum samples from ASD and TD males. These included thyroid stimulating hormone (TSH), interleukin 8 (IL-8) and monokine induced by gamma interferon (MIG). In the present study TSH levels were significantly lower in the ASD boys compared to TD boys. From the random forest analysis, TSH had the highest importance among the panel of 11 analytes for predicting ASD vs. TD. When TSH was studied on the MSD platform, again the ASD boys had an average 26% lower level vs. TD boys (see Table 2). It is interesting that the ASD girls did not exhibit a decrease in TSH vs. TD girls. This finding is consistent with several studies reporting sex-specific differences in putative ASD biomarkers (Schwarz et al., 2011; Spratt et al., 2014 and Steeb et al., 2014), but the inventors' finding requires further study because of the low sample size for the female group.

TSH is a pituitary hormone that stimulates the thyroid gland to produce thyroxine (T₄), and then triiodothyronine (T₃), which stimulates the metabolism of almost every tissue in the body (Morreale de Escobar et al., 2004). TSH is secreted throughout life but reaches high levels during the periods of rapid growth and development. The hypothalamus produces thyrotropin-releasing hormone (TRH), which stimulates the pituitary gland to produce TSH. Thyroid hormones are essential for brain maturation, and for brain function throughout life. Thyroid hormone deficiency, even of short periods may lead to irreversible brain damage, the consequences of which depend on the specific timing of onset and duration of thyroid hormone deficiency (Anderson et al., 2003; Bernal, 2005; Koibuchi & Iwasaki, 2006; Morreale de Escobar et al., 2004).

Reductions in TSH have been reported previously for ASD children. Significantly reduced levels of TSH, and reduced TSH response following TRH stimulation, have been observed by Hashimoto et al. (1991) where they examined 41 ASD boys (average of 5.7 yrs) compared to 5 TD boys (average of 8.7 years). They also examined 12 boys with mental retardation and 12 boys with minimal brain dysfunction, and their TSH levels were like that of the TD boys. More recently, the RBM platform was used to demonstrate altered levels of 15 blood proteins, and one of the proteins was TSH (Mizejewski et al., 2013). Reduced levels of TSH were observed in bloodspots from infants who later were found to have ASD (n=16 ASD and n=32 TD; gender not reported). These data indicate that the reduced TSH was present at birth. More recently, maternal mid-pregnancy serum TSH levels were found to be inversely correlated with the likelihood of having a child with ASD (Yau et al., 2015). This study used 149 control children and 78 ASD children, and both genders were in the two groups.

Proinflammatory cytokines (PICs) have been reported to be elevated in ASD children (e.g., Ashwood et al., 2011). In the present study using both the RBM and MSD platforms, the inventors found 15-30% increases in IL-8 in the serum of ASD boys. In the study of Mizejewski et al. (2013), elevations in IL-8 were reported in ASD children in bloodspots collected at birth. PICs are directly linked with neuroinflammation. Elevations in plasma IL-8 (33%) have also been reported by Suzuki et al. (2011) in high-functioning ASD boys, with a mean age of 12 years (n=28 ASD boys and N=28 TD boys).

The present results add to a growing literature to indicate that chemokines, cytokines and hormones are abnormal, apparently from birth, in boys with ASD. These findings also are of interest because the changes in TSH in ASD boys was not observed in ASD girls, consistent with other observations that the disorder differs with gender (Schwarz et al., 2011; Spratt et al., 2014 and Steeb et al., 2014). These data suggest a certain common immunological pathology among boys with ASD.

There are a number of limitations in this study. The small sample size renders the data presented here preliminary, and a larger study with more ASD subjects is underway. However, since recruitment for the current study was limited to ASD subjects with a diagnosis of autistic disorder, none of whom were taking psychotropic drugs, the inventors could control for some confounding factors. The increased prevalence of ASD in boys resulted in the study primarily focusing on boys, which does not allow one to thoroughly investigate gender-specific differences. However, the data from this study suggest that even with small sample sizes, gender-specific differences are evident. This should be further evaluated in a larger study. When making electrochemiluminescent measurements on 96-well plates, there are often plate-to-plate differences that add variability to the data; however, the inventors routinely run standards on every plate as well as calibration curves to minimize this source of data variability.

In order to identify ASD at an early age, to facilitate treatment before symptoms manifest, biomarkers are important. It is interesting that when the inventors analyzed the accuracy of predicting ASD vs. TD using more than one analyte, they found that the accuracy went from 67-69% for single analytes, to 76.7% when using the two analytes together. The use of panels of blood proteins for disease identification appears to be a useful strategy and one that the inventors will pursue by testing additional analytes identified in the RBM analysis (e.g., chromogranin A, ferretin, apolipoproteins E and H) to determine whether 5-7 of the protein analytes combined will provide a sensitivity/specificity of ˜90% in predicting ASD in boys.

### Example 2

### Materials and Methods

Human Subjects.

The study protocol and all subsequent amendments were submitted by The Johnson Center for Child Health and Development (Austin, Tex.) and approved by the Austin Multi-Institutional Review Board (AMIRB) for ASD and TD subjects, and the IRB at UT Southwestern (UTSW) Medical School for adult subjects.

The ASD group was comprised of 50 male subjects with a median age of 5.6 years (range—2.3-9.5 years). The TD group was comprised of 43 males with a median age of 6.2 years (range—2.5-9.5 years). The inventors also used 10 ASD females (median age 5.3 and 20 TD females (median age 2.2-7.5. These subjects were either recruited directly from The Johnson Center clinic, or through the use of informational study flyers. Written informed consent was received from the parent or guardian of all subjects prior to enrollment. For the ASD group, all subjects were assessed by The Johnson Center clinical psychologist using both the Autism Diagnostic Observation Schedule (ADOS) and the Autism Diagnostic Interview-Revised (ADI-R). Clinical diagnosis was made based on these data and overall clinical impression using DSM-IV criteria. For this particular study, subjects with a diagnosis of Asperger's Syndrome or Pervasive Developmental Disorder—Not Otherwise Specified (PDD-NOS), were excluded. For the TD group, all subjects underwent a developmental screening using the Adaptive Behavior Assessment System—Second Edition (ABAS-II). TD subjects were excluded if their score on the ABAS-II suggested possible abnormal development and the need for further evaluation. TD subjects were also excluded if they had any first- or second-degree relatives diagnosed with ASD. Any subjects diagnosed with a genetic, metabolic, or other concurrent physical, mental, or neurological disorder were also excluded, as were any subjects that were currently taking psychiatric medications (or had taken psychiatric medications within the last 3 months prior to enrollment).

Normal control older adult male serum samples (n=53) were obtained from the UTSW Alzheimer's Disease Center and the Parkinson's Disease Biomarker Program. All subjects were cognitively normal and free from neurodegenerative diseases based upon clinical evaluation, neuropsychological testing and in some cases brain scans. The median age of these subjects was 69 years (range 40-75 years).

Blood Collection and Storage.

A fasting blood draw was performed on ASD and TD subjects between the hours of 8-10 a.m. Blood was collected into a 3.5 ml Serum Separation Tube (SST; Vacutainer System; Becton-Dickinson) using standard venipuncture technique. The blood was gently mixed in the SST by 5 inversions and then stored upright for clotting at room temperature for 30 mins. Blood was spun immediately after the clotting time in a swing bucket rotor for 20 minutes at 1100-1300 g at room temperature. Serum was removed immediately after centrifugation and transferred into coded cryovials in 0.25 ml aliquots. Aliquots of serum were immediately placed upright in a specimen storage box in a −20° C. freezer for up to 6 hours. Samples were then transferred to a −80° C. freezer for long-term storage. Sample aliquots were shipped to UTSW on dry ice. The blood from adult male control subjects was collected and stored according to protocols established by the Alzheimer's Disease Neuroimaging Initiative (see world-wide-web at adni-info. org/Scientist/Pdfs/adni_protocol 9 19 08.pdf).

Peptoid Library Synthesis.

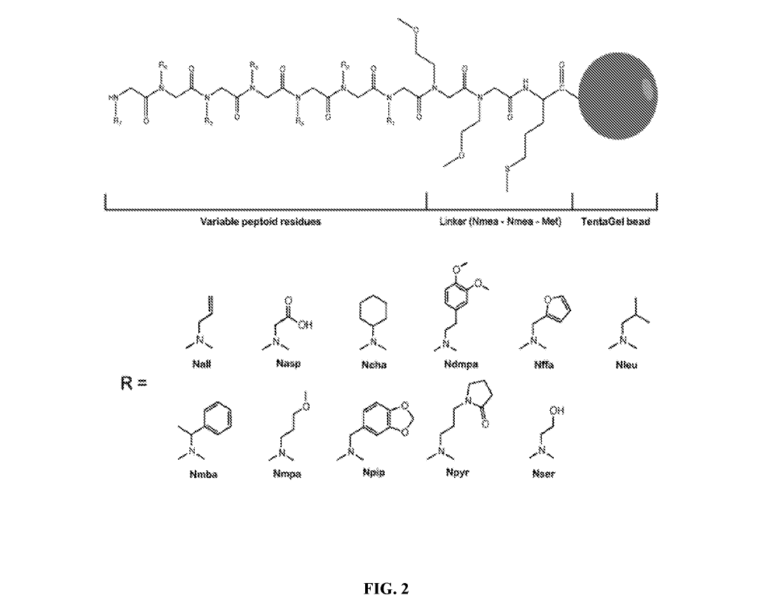

Three distinct one-bead one-compound combinatorial libraries of peptoids (oligo-N-substituted glycines) were synthesized onto 75 μm TentaGel beads using a split and pool method (Figliozzi et al., 1996). Library 1 was configured as NH2-X7-Nmea-Nmea-Met-TentaGel, where X=Nall, Nasp, Ncha, Nffa, Nleu, Nmba, Nmpa, Nphe, Npip, or Nser, yielding a theoretical diversity of 10⁷ possible compounds (FIG. 2). (Monomer abbreviations: Met=methionine, Nall=allylamine, Nasp=glycine, Nbsa=4-(2-aminoethyl)benzenesulfonamide, Ncha=cyclohexylamine, Ndmpa=3,4-dimethoxyphenethylamine, Nffa=furfurylamine, Nippa=3-isopropoxypropylamine, Nleu=isobutylamine, Nlys=1,4-diaminobutane, Nmba=(R)-methylbenzylamine, Nmea=2-methoxyethylamine, Nmpa=3-methoxypropylamine, Nphe=benzylamine, Npip=piperonylamine, Npyr=N-(3′-aminopropyl)-2-pyrrolidinone, Nser=ethanolamine). Library 2 was configured as NH2-X6-Nmpa-Nlys-Met-TentaGel, where X=Nall, Nasp, Ncha, Nippa, Nleu, Nlys, Nmba, Npip, Npyr, Nser (theoretical diversity=10⁶ possible compounds). Library 3 was configured as NH2-X5-Nmea/Nlys-Ndmpa-Nmea-Met-TentaGel, where X=Nall, Nasp, Nbsa, Nippa, Nleu, Nlys, Nmba, Npip, Npyr, Nser (theoretical diversity=200,000 possible compounds). Methionine linkers were coupled in the usual way, while the peptoid residues were coupled using the submonomer method (Figliozzi et al., 1996) with microwave irradiation to accelerate reactions (Olivos et al., 2002). Proper library syntheses were confirmed by CNBr cleavage of compounds from samples of isolated beads and subsequent analysis by tandem mass spectrometry.

On-Bead Magnetic Screening.

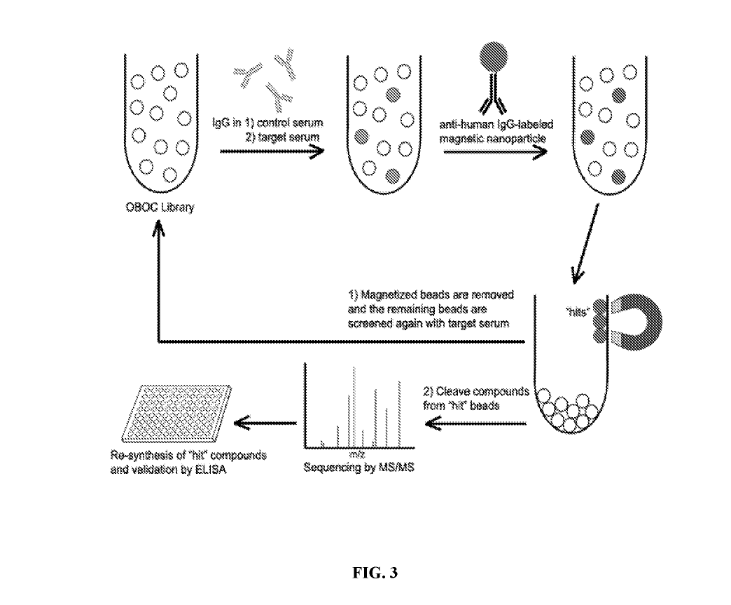

A modification of the magnetic capture method for screening on-bead libraries (Olivos et al., 2002) was used. Approximately 375,000 beads from the library were soaked in PBST (PBS-0.1% Tween 20, pH 7.4) and then blocked with blocking buffer (1:1 mixture of 1% bovine serum albumin (BSA) in PBST and SuperBlock Blocking Buffer (Thermo Scientific, Rockford, Ill.)) for 1 hr at room temperature (RT). Serum aliquots from 10 TD males were pooled and then diluted up to 1 ml in blocking buffer to obtain a final IgG concentration of 40 μg/ml. IgG levels of serum pools were measured using Human IgG ELISA Quantitation Set (Bethyl Laboratories, Montgomery, Tex.). The library beads were then incubated with the diluted serum in a cryovial overnight at 4° C. The beads were washed with PBST eight times and re-suspended in blocking buffer. A 10 mg/ml anti-human IgG-conjugated Dynabead solution, 50 μl, was prepared by coupling 10 μg of biotinylated Goat F(ab)₂ anti-human IgG (Southern Biotech, Birmingham, Ala.) with 0.5 mg of Dynabead M-280 Streptavidin (Invitrogen, Grand Island, N.Y.). The library beads were mixed with the Dynabead solution for 2 hrs at room temperature (RT) with gentle agitation. Library beads with high levels of bound Dynabeads were then separated by placing the tube in a strong magnetic field. These “magnetized” beads were removed from the library. The remaining beads were again washed and the magnetic capture was repeated two more times, completing the depletion. The depleted library was then incubated with pooled serum aliquots from 10 ASD males as described above. “Hit” beads were obtained by performing the magnetic capture and collecting the magnetized beads. “Hit” peptoid compounds were then identified by CNBr cleavage of compounds from “hit” beads and sequencing by MALDI TOF/TOF (FIG. 3).

For validation and subsequent analyses, “hit” peptoid compounds were re-synthesized on Polystyrene AM RAM resin (Rapp Polymere, Tubingen, Germany) with the methionine linker, as in the library, replaced by a cysteine linker so that the compounds could be immobilized using sulfhydryl-reactive chemistry. Peptoid compounds were cleaved off the resin by incubating in a 95% trifluoroacetic acid, 2.5% triethylsilane, 2.5% water solution for 2 hrs at RT. Peptoid compounds were subsequently purified using high-performance liquid chromatography and verified by MS analysis.

Peptoid ELISA.

Peptoid compounds were immobilized onto maleimide-activated 96-well plates (Pierce Biotechnology, Rockford, Ill.) by dissolving them to 0.03-0.05 mM in a 0.1 M sodium phosphate, 0.15 M sodium chloride, 10 mM EDTA solution adjusted to pH 7.2 and incubating with shaking for 3 hrs at RT. Plates were washed with PBST and blocked with a 5% goat serum (Thermo Scientific, Rockford, Ill.) in PBST solution for 1 hr at RT. Plates were washed again and incubated with target (serum) samples diluted in blocking buffer (1:1 PBST-1% BSA and SuperBlock) for 2 hrs at RT. After washing, plates were incubated with Goat anti-Human IgG-Fc-HRP conjugate (Bethyl Laboratories, Montgomery, Tex.) diluted 1:30,000 in PBST-1% BSA or mouse anti-human IgG1 (hinge)-HRP conjugate (SouthernBiotech, Birmingham, Ala.) diluted 1:1000 in PBST-1% BSA for 30 min at RT. After another wash, plates were incubated in TMB substrate for 16 min at RT and stopped with 2M H₂SO₄. Plates were read at 450 nm. All samples were run in duplicate, and every assay contained ASD and TD serum pool samples to serve as internal controls. Results for individual samples were assessed as ratios to the ASD serum pool so as to control for plate-to-plate variation.

For control experiments, total IgG levels for individual serum samples were quantified using human IgG ELISA Quantitation Set (Bethyl Laboratories, Montgomery, Tex.), and IgG1 levels were quantified using IgG Subclass Human ELISA Kit (Invitrogen, Grand Island, N.Y.).

Affinity Purification of Peptoid-Binding Proteins.

The ASD1 peptoid was coupled to iodoacetyl gel columns from MicroLink Peptide Coupling Kit (Thermo Scientific, Rockford, Ill.) at concentrations of 3-5 mM. Regarding the step requiring blocking of excess iodoacetyl groups with L-Cysteine, identical protein bands were observed in preliminary gels with or without the inclusion of this step. Therefore, the step was omitted in subsequent iterations. ASD1-coupled gel columns were then incubated with 1:60 dilutions of serum in PBST for 2 hrs at RT with gentle agitation. Flow-through was discarded and peptoid-binding proteins were collected by elution with 100 μl of the low-pH buffer provided. The inventors used 5 μl of 1M Tris, pH 9.0 added to each eluted aliquot to neutralize the low pH. The eluted aliquots were then each lyophilized and re-dissolved in 10 μl of PBS to maximize the protein concentration for visualization on the gel.

Gel electrophoresis and Coomassie Blue staining. Peptoid-binding protein analytes were loaded into 4-20% Mini-Protean TGX Precast gels (Bio-Rad Laboratories, Hercules, Calif.) after mixing with 5 μl 10% 2-mercaptoethanol in Laemmli Sample Buffer (Bio-Rad Laboratories, Hercules, Calif.) and heating at 95° C. for 5 min. After electrophoresis, the gel was stained with 0.1% Coomassie Blue R-250 (Bio-Rad Laboratories, Hercules, Calif.) in a 10% acetic acid, 50% methanol, 40% water solvent for 2 hrs at RT with gentle agitation. Gels were then destained overnight at RT in the same solvent with gentle agitation and two changes of solvent throughout.

Identification and Validation of “Hit” Peptoids.

In an effort to isolate peptoid compounds that bind antibodies specific to children with ASD versus TD, three distinct one-bead one-compound peptoid libraries were synthesized and screened using serum pools from both groups. The first library consisted of 10-mer compounds with 7 variable peptoid residues that could be any of 10 different amines, yielding a theoretical diversity of 10⁷ possible compounds (FIG. 2). During screening, the library was first depleted of peptoids that bound IgG in serum pooled from 10 TD males. The depleted library was then incubated with serum pooled from 10 ASD males. Compounds that were then found to bind IgG were designated as “hit” peptoids (FIG. 3). A second library was then synthesized in an attempt to reduce the large number of nonspecific “hit” beads yielded during screens with the first library. This library was designed to be less hydrophobic in character by the inclusion of the charged residue, Nlys (diaminobutane), in both the invariant linker and as a possible amine in the variable region. Finally, a third library was synthesized with a theoretical diversity lower than the previous two—only 200,000 possible compounds—to encourage the isolation of redundant compounds during screening as an intermediate measure for validating the specificity of “hits” (Olivos et al., 2002). Screens of these latter libraries were performed with the same serum pools in the same way as with the first.

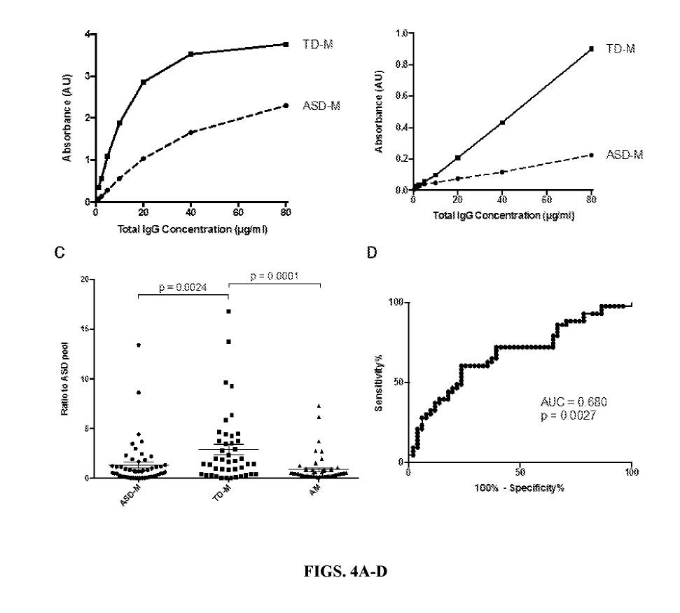

Using the same two serum pools as used during screening, the “hit” peptoids were then tested by ELISA for their ability to bind IgG from the two pools. Based on the screening design, and as observed in previous studies with other sample sets (Olivos et al., 2002; Olivos et al., 2002 and Olivos et al., 2002), it was expected that successful validation of a “hit” peptoid would demonstrate the compound to bind higher levels of IgG from the ASD pool than from the TD pool. In total, 25 “hit” peptoids were isolated from all three libraries, identified, re-synthesized, and tested in this way by ELISA. None of them, however, satisfied the expectation for a successful validation. Rather, all of them, independent of the library from which they were isolated, uniformly demonstrated the opposite pattern of binding in which they bound higher levels of IgG from the TD pool than from the ASD pool. The binding pattern of one of these compounds isolated from the first library and named ASD1 is shown in FIG. 4A.

Statistics. All statistical analyses were performed using GraphPad Prism 6. The mean values of un-transformed ELISA data for individual samples were compared by Analysis of variance.

### Results

Assessment of ASD1.

Although the 25 compounds isolated from the various screens did not demonstrate the ability to bind antibodies elevated in ASD, they were able to consistently differentiate between the ASD and TD serum pools in levels of IgG binding. The ASD1 peptoid was chosen to further assess the nature of this differentiation. FIG. 4B shows how the differentiation in binding between the serum pools can be amplified from two-fold to nearly four-fold using a secondary antibody specific to the IgG1 subclass. Using this same secondary antibody, serum samples from many individual ASD (n=50) and TD (n=43) male subjects were tested against ASD1 (FIG. 4C). Consistent with the serum pool results, ASD1 binding of IgG1 from TD males (n=43; 2.88±0.55) was significantly higher than that of IgG1 from ASD males (n=50; 1.33±0.31; p=0.0024). The receiver-operating characteristic curve for discriminating ASD vs. TD was 0.680, p=0.0027 (FIG. 4D). The inventors also examined ASD1 binding to a small sample of females; the binding to ASD (n=10) and TD females (n=20) was 1.76±0.81 and 1.97±0.38, respectively which is intermediate to the values found in ASD and TD males.

The inventors also compared binding of the ASD1 peptoid to serum samples from older adult males of mean age 66.7 years (n=53). ASD1 binding of IgG1 from the older adult males did not differ from that of the ASD males, but was significantly lower than that of the TD males (p=0.0001) (FIG. 4C).

Isolation of ASD1-Binding Proteins.

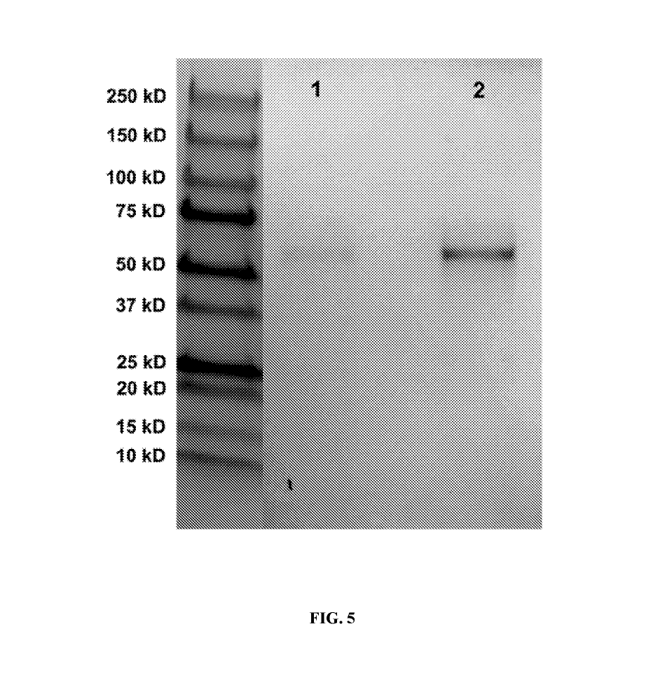

In an attempt to identify the antibody(-ies) recognized by ASD1, affinity purification with ASD1 was performed against the same ASD and TD serum pools used during screening. The proteins left bound to ASD1 were eluted out and analyzed by gel electrophoresis and Coomassie Blue staining. A single band was observed whose molecular weight ˜55-60 kD (FIG. 5). The band is observable in both ASD and TD analytes, and its intensity is higher in the TD analyte, which correlates with the IgG1 binding to ASD1 observed with ELISA.

### Discussion

Peptoid libraries have been used previously to search for autoantibodies for neurodegenerative diseases (Reddy et al., 2011) and for systemic lupus erythematosus (SLE) (Quan et al., 2014). In the case of SLE, peptoids were identified that could identify subjects with the disease and related syndromes with a goo sensitivity (70%) and excellent specificity (97.5%). Peptoids were used to measure IgG levels from both healthy subjects and SLE patients. Binding to the SLE-peptoid was significantly higher in SLE patients vs. healthy controls. The IgG bound to the SLE-peptoid was found to react with several autoantigens, suggesting that the peptoids are capable of interacting with multiple, structurally similar molecules. These data indicate that IgG binding to peptoids can identify subjects with high levels of pathogenic autoantibodies vs. a single antibody.

In the present study, the ASD1 peptoid binds significantly lower levels of IgG1 in ASD boys vs. TD boys. This finding indicates that the ASD1 peptoid recognizes an IgG1 subtype that is significantly lower in abundance in the ASD boys vs. TD boys. These data are consistent with the observation (Heuer et al., 2008) that ASD children (n=116) have >30% lower levels of plasma IgG compared to TD children (n=96) in the same age range as studied here. In the small sample of ASD and TD females the ASD1 binding did not differ between the two groups and the binding level was intermediate between that observed in the ASD vs. TD males.

The peptoid protocol used to identify disease-related antibodies generally expects to identify antibodies that are higher in abundance in the disease group vs. the normal control group. This is because the peptoid library is first screened with blood from a pool containing normal control subjects and the peptoids that bind high levels of IgG are removed from the library. When the disease pool is screened next, it is exposed to peptoids that are not expected to recognize IgGs that are normally in high abundance. Thus, the peptoid identifies IgGs that are in higher abundance in the disease group vs. the control group (Reddy et al., 2011 and Quan et al., 2014). In the present study, the opposite case was observed; lower levels of IgG binding were found in the ASD group vs. normal control group (TD males). This observation was made for 25 different ASD-related peptoids. For ASD, it appears that because the ASD serum generally binds low levels of IgGs, no matter how many high-abundance peptoids are pulled out of the library using pooled control serum, the normal control group will always bind higher levels of IgG compared to the ASD group.

It is interesting that in serum samples from older men, the ASD1 binding is similar to that in the ASD boys. This is consistent with the observation that with aging there is a reduction in the strength of the immune system, and the changes are gender-specific (Gubbels Bupp, 2015). Recent studies using parabiosis (Villeda et al., 2014), in which blood from young mice reverses age-related impairments in cognitive function and synaptic plasticity in old mice, reveal that blood constituents from young subjects may contain important substances for maintaining neuronal functions. Work is in progress to identify the antibody/antibodies that are differentially binding to the ASD1 peptoid, which appear as a single band on the electrophoresis gel.

There are a number of limitations of this study. The relatively small sample size renders the data presented here preliminary, and a larger study with more ASD subjects is underway. However, since recruitment for the current study was limited to ASD subjects with a diagnosis of autistic disorder, none of whom were taking psychiatric medications, the inventors could control for some potential confounding factors. The increased prevalence of ASD in boys resulted in the study group including more boys than girls, which does not allow one to thoroughly investigate any gender-specific differences. However, the data from this study suggest that even with small sample sizes, gender-specific differences appear to be present. This observation should be further evaluated in a larger study.

Blood biomarkers for diseases have become a topic of great recent interest. Such biomarkers are relatively non-invasive for the patient and inexpensive to analyze. Panels of biomarkers have been used to identify Alzheimer's disease (AD); panels consisting of 10-20 proteins have been identified which allow ˜90% sensitivity and specificity for the identification of AD (O'Bryant et al., 2014), and panels of plasma phospholipids have proven useful for the identification of AD (Mapstone et al., 2014), and even panels of pathogenic proteins (i.e., amyloid (31-42 and P-T181 tau) (Fiandaca et al., 2014). For ASD, biomarker searches are relatively new. A study using metabolomics (West et al., 2014) identified a panel of >100 mass feature to produce an optimal predictive signature for ASD in a small sample ASD and TD children. The accuracy of prediction for this panel was 81%. More recently, a blood-based panel of genomic biomarkers was identified for young boys with ASD (Pramparo et al., 2015). The accuracy of this panel was 75%. This signature of differentially expressed genes was enriched in translation and immune/inflammation functions. When the inventors combined ASD1 binding with thyroid stimulating hormone levels in the same ASD and TD boys, they find a predictive accuracy of 73% as opposed to 66% for the ASD1 peptoid alone. Because panels of blood proteins can provide a high sensitivity/specificity for identifying a disease, the inventors are studying whether the ASD1 peptoid in combination is inflammatory blood analytes can serve as a useful biomarker panel for ASD.

### Example 3

### Methods

Ethics.

The study protocol and all subsequent amendments were submitted by The Johnson Center for Child Health and Development (Austin, Tex.) and approved by the Austin Multi-Institutional Review Board (AMIRB) for ASD and typically developing (TD) subjects.

Study Subjects.

The ASD and TD groups were comprised of male subjects age 2 to 8 years of age. Subjects were either recruited directly from The Johnson Center clinic, or through the use of informational study flyers circulated around Austin, Tex. Written informed consent was received from the parent or guardian of all subjects prior to enrollment. Briefly, the psychiatric, medical, and family histories of all participants were obtained. For the ASD group, the subjects were assessed by a psychologist trained in research reliability using both the Autism Diagnostic Observation Schedule (ADOS) and the Autism Diagnostic Interview-Revised (ADI-R). Clinical diagnosis was made based on these data and overall clinical impression using DSM-IV criteria. For this particular study, subjects with a diagnosis of Asperger's Syndrome or Pervasive Developmental Disorder—Not Otherwise Specified, were excluded. For the TD group, all subjects underwent a developmental screening using the Adaptive Behavior Assessment System-Second Edition (ABAS-II) that was assessed by the psychologist. TD subjects were excluded if their score on the ABAS-II suggested possible abnormal development, and the need for further evaluation. TD subjects were also excluded if they had a first- or second-degree relative diagnosed with ASD. Subjects diagnosed with a genetic, metabolic, or other concurrent physical, mental, or neurological disorder, were excluded, as were subjects that were currently taking psychiatric medications (or had taken psychiatric medications within the last 3 months prior to enrollment). All subjects were healthy with no reported illnesses for 3 weeks prior to participation in the study.

Due to the high degree of phenotypic heterogeneity in ASD, the inventors further sub-characterized ASD subjects into three groups (Napolioni et al., 2013): (i) those who were non-verbal, (ii) those with gastrointestinal (GI) concerns; and (iii) those with regressive autism. Subjects with ASD were defined as non-verbal if there was a complete absence of intelligible words at time of diagnostic assessment of autism. ASD subjects were classified as having GI concerns if they reported at least one of the following symptoms: (i) constipation, (ii) diarrhea, (iii) abdominal bloating, discomfort, or irritability, (iv) gastroesophageal reflux or vomiting, and/or (v) feeding issues or food selectivity. ASD subjects were classified as having no-regression if the child exhibited traits of autism from infancy, and regressive autism if they had typical early development and later lost function in language and/or social interactions (based on questions probed in the ADI-R). The correlations between protein levels, phenotypic sub-groups, and clinically relevant quantitative traits from the ADOS were analyzed.

Blood Collection/Storage.

A fasting blood draw was performed on healthy children between the hours of 8-10 a.m. Blood was collected in a 3.5 ml Serum Separation Tube (SST; Vacutainer System; Becton-Dickinson) using standard venipuncture technique. The blood was gently mixed in the SST by 5 inversions and then stored upright for clotting at room temperature for 10-15 min. Blood was then spun immediately after the clotting time in a swing bucket rotor for 15 min. at 1100-1300 g at room temperature. Serum was removed immediately after centrifugation and transferred into coded cryovials in 0.5 ml aliquots. Aliquots of serum were immediately placed upright into storage boxes in a −20° C. freezer for up to 6 hours. Samples were then transferred to a −80° C. freezer for long-term storage.

Analyte Measurements on Rules Based Medicine Platform.

Sample aliquots were coded to remove the possibility of bias and shipped on dry ice to Myriad-Rules Based Medicine (RBM; Austin Tex.) for evaluation using DiscoveryMAP 175+ for quantitative immunoassay of inflammatory molecules and hormones. A total of 30 ASD and 30 TD male serum samples were analyzed. A multianalyte Luminex profiling platform is used containing over 175 protein analytes. Final data were reported as the absolute concentrations in the serum. Some coded duplicate samples were run and used to analyze analyte measurement performance. Those analyte measurements that showed >15% variance were excluded from the data analysis.

Validation Measurements on the Meso Scale Discovery Platform.

The inventors sought to begin to validate the serum biomarker proteins identified on the RBM platform using a different platform, which was run in their lab. Compared with the traditional ELISA approach, the Meso Scale Discovery (MSD) platform shows greater sensitivity and is able to reliably detect different proteins across a broad dynamic range of concentrations (Burguillos, 2013). The assay is based upon electrochemiluminescence technology by using specific capture antibodies coated at corresponding spots on an electric wired microplate. This platform was used to measure TSH in 43 ASD boys and 37 TD boys, and IL-8 in 36 ASD boys and 35 TD boys; the two proteins showing the greatest percent difference in the ASD and TD samples run on the RBM platform. The inventors measured the two proteins in samples that were run in duplicate accordingly to the manufacturer's protocol. Any duplicate value with >15% variance was removed from the final data analysis, and every plate was run with a standard concentration curve.

Statistical Analyses.

The RBM data were analyzed using Random Forest methods. Random forest analysis was developed as an ensemble learning method that utilizes a classification tree as the base classifier (Breiman, 2001). Hundreds of training and test sets, of 15 subjects/group, were analyzed to determine the importance of a panel of analytes to correctly identifying ASD subjects. For the MSD data, differences between the ASD and TD groups were analyzed with Mann Whitney U-tests. For comparing the accuracy of two analytes for predicting ASD vs. TD, the inventors used cutscores, and area under the curve (AUC) analyses. Diagnostic accuracy and AUC were computed by ROC (Receiver Operation Characteristic) curves using SPSS V23; the optimum probability cutoffs were determined using mathematical formulas in Microsoft Excel™ to maximize accuracy and the perpendicular distance from the 45 degree line of equality. The p<0.05 level was considered to be statistically significant for analyses using the MSD platform (i.e., for TSH and IL-8 assays).

Regression analyses for ASD subjects were conducted using the R lavaan package, which fits models using full information maximum likelihood estimation that makes use of all available data. Thus, data from all ASD subjects were included in each model (Graham, 2012) (n=43 for THS and n=36 for IL-8). Protein levels were regressed on each of the ADOS subdomain scores and phenotypic sub-grouping to examine whether levels of THS and/or IL-8 were related to a clinical measure of ASD and comorbidities. Prior to fitting regression models, IL-8 was log-transformed to reduce the positive skew; the transformed distribution was approximately normally distributed and met guidelines for covariance matrix based models (Curran et al., 1996).

### Results

Proteins Measured on the RBM Platform.

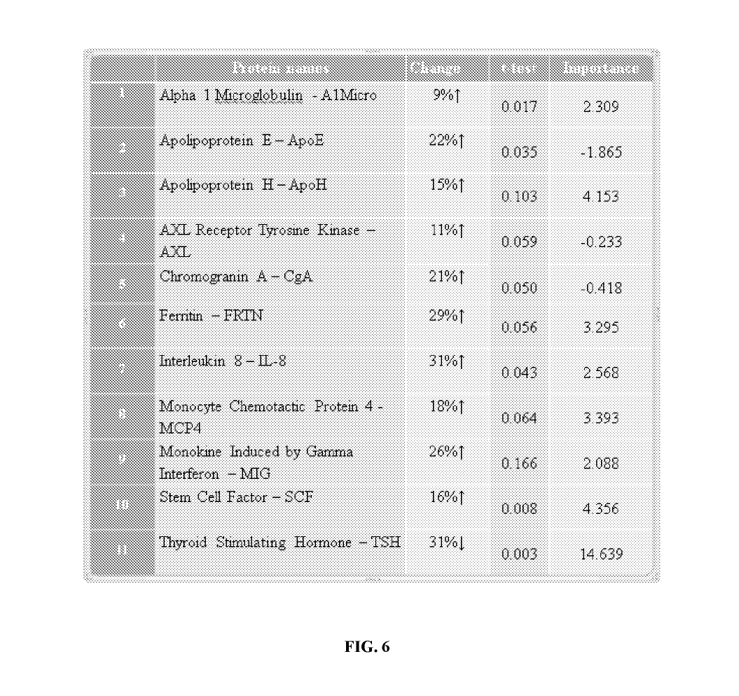

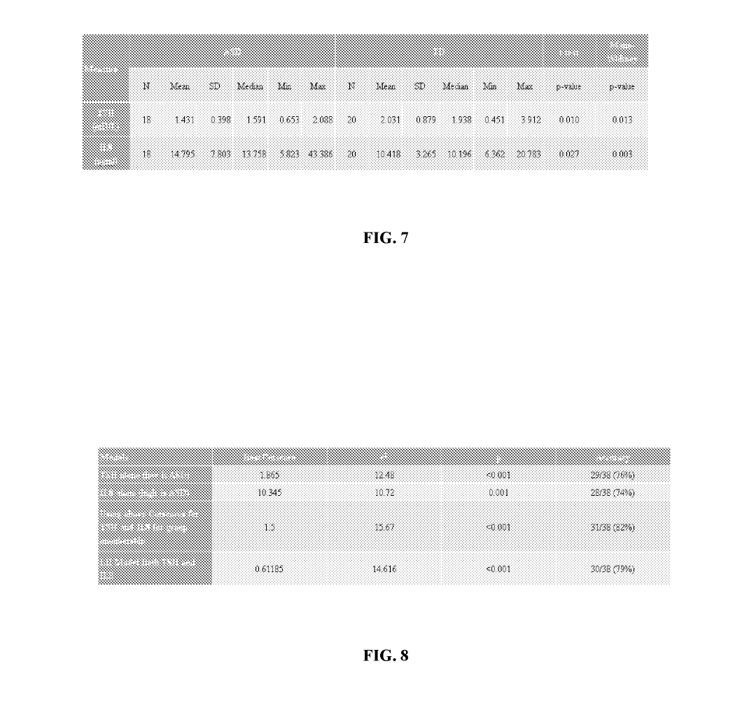

A total of 184 analytes were measured on the RBM luminex platform, however 51 were undetectable, and 23 exhibited >15% spot-to-spot variance and were therefore omitted from analysis. The undetectable proteins likely represent faulty antibodies, as these proteins have been detected with previous versions of DiscoveryMap. Eleven of the remaining 110 serum proteins measured, shown in FIG. 6, were selected based upon a Random Forest analysis, and 5 of the analytes were significantly different between ASD and TD at the p≤0.04 level (uncorrected for multiple comparisons). The Random Forest Test-sets ability to accurately predict ASD vs. TD most often exhibited areas under the ROC curves≥0.60.

Proteins Measured on the MSD Platform.

The inventors chose to measure, on the MSD platform, levels of two of the proteins identified on the RBM platform that showed the greatest percent difference between the ASD and TD groups—TSH and IL-8. All of the samples run on the RBM platform were also run on the MSD platform, plus some additional samples were included to increase the sample size.

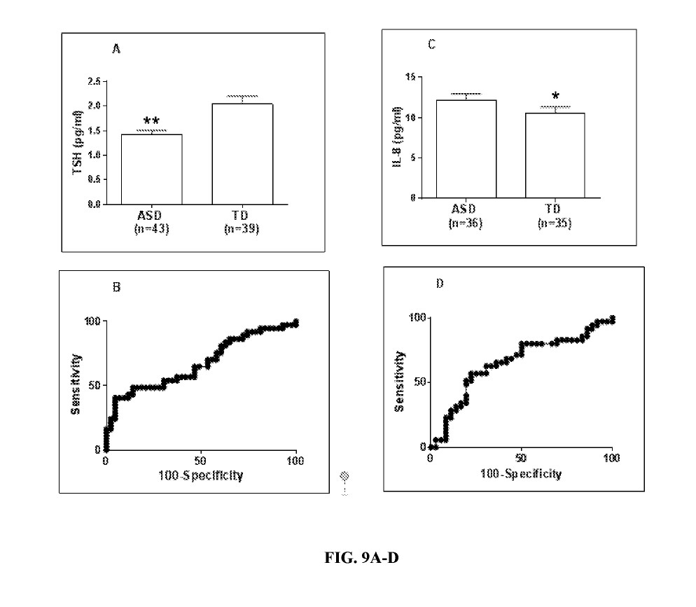

TSH levels were 30% lower in ASD boys (n=43) vs. TD boys (n=37): 1.42±0.08 (mean±SEM) and 2.04±0.16 mIU/1, respectively (p<0.0056; Mann Whitney U test). (see FIG. 9). The area under the ROC curve is 0.674, p=0.006. IL-8 levels were 16% higher in ASD boys (n=36) vs. TD boys (n=35): 12.17±0.52 (mean±SEM) and 10.52±0.51 pg/ml, respectively (p<0.0306; Mann Whitney U test) (see FIG. 9). The area under the ROC curve is 0.652, p=0.023.

In order to determine whether the accuracy in predicting ASD vs. TD is enhanced by an analysis using a combination of analytes, the inventors analyzed the prediction accuracy using both TSH and IL-8 analytes (see FIG. 8). Here, they used samples from subjects that were run for both analytes on the MSD platform, which resulted in 18 ASD and 20 TD boys. The predictive Accuracy for TSH alone was 76% and for IL-8 alone it was 74%. Using the two analytes together the predictive Accuracy was 82%, with 88% Sensitivity and 75% Specificity. ASD cases were predicted, using cutscores, as having TSH levels below 1.8 and IL-8 levels above 10.3. The area under the curve for the model using the two analytes was 0.842±0.067 SEM (p<0.001).

Association Between Protein Levels and ADOS Subdomains.

TSH was regressed on each of the ADOS subdomain scores. The following three domains exhibited a significant negative correlation whereby higher scores in the subdomains were associated with lower levels of TSH: Social Interaction (z=−2.61, p=0.009), Communication+Social Interaction (z=−2.12, p=0.034), and Stereotyped Behavior and Restrictive Interests (SBRI) (z=−2.28, p=0.023). There was not a significant relationship between TSH and the ADOS Communication subdomain (z=−0.55, p=0.581).

IL-8 was regressed on each of the ADOS subdomain scores. Among ADOS subdomains, there were no significant relationships between IL-8 and Communication (z=0.16, p=0.871), Social Interactions (z=−0.15, p=0.877), Communication+Social Interactions (z=−0.06, p=0.953), or SBRI (z=0.75, p=0.455).

Association Between Protein Levels and Phenotypic Data.

The percentage of children with ASD who were nonverbal was 50%. The percentage of children with ASD displaying GI issues was 85%. Regressive autism was seen in 63% of the study group. There were no significant relationships between either TSH or IL-8 and the autism sub-groups. For TSH: Non-Verbal (z=−0.51, p=0.609), GI concerns (z=−0.14, p=0.890), and Regression (z=−1.12, p=0.265). For IL-8: Non-Verbal (z=−0.20, p=0.843), GI concerns (z=−0.21, p=0.833), and Regression (z=−0.49, p=0.624).

Using a quantitative immunoassay for inflammatory molecules and hormones, 11 proteins were found, when combined, to discriminate serum samples from ASD and TD males. Among these 11 proteins TSH, IL-8, alpha 1 microglobulin, apolipoprotein E, and stem cell factor exhibited the highest significant differences between the two groups. TSH levels were significantly lower in the ASD boys compared to TD boys, and based upon the Random Forest analysis, TSH had the highest Importance among the panel of 11 analytes for predicting ASD vs. TD. When TSH was studied on the MSD platform, again the ASD boys had an average 30% lower level vs. TD boys. The inventors limited this study to boys because several studies report sex-specific differences in putative ASD biomarkers (Schwarz et al., 2011, Sprott et al., 2014 and Steeb et al., 2014), and the inventors only had access to a small sample from ASD and TD girls.

TSH is a pituitary hormone that stimulates the thyroid gland to produce thyroxine (T₄), and then triiodothyronine (T₃), which stimulates the metabolism of almost every tissue in the body (Morreale et al., 2004). TSH is secreted throughout life but reaches high levels during the periods of rapid growth and development. The hypothalamus produces thyrotropin-releasing hormone (TRH), which stimulates the pituitary gland to produce TSH. Thyroid hormones are essential for brain maturation, and for brain function throughout life. Thyroid hormone deficiency, even of short periods may lead to irreversible brain damage, the consequences of which depend on the specific timing of onset and duration of thyroid hormone deficiency (Morreale et al., 2004, Anderson et al., 2003, Bernal, 2005 and Koibuchi and Iwasaki, 2006).

Reductions in TSH have been reported previously for ASD children. Significantly reduced levels of TSH, and reduced TSH response following TRH stimulation, have been observed by Hashimoto et al. (Hashimoto et al., 1991), where they examined 41 ASD boys (average of 5.7 yrs) compared to 5 TD boys (average of 8.7 years). They also examined 12 boys with mental retardation and 12 boys with minimal brain dysfunction, and their TSH levels were like that of the TD boys. More recently, the RBM platform was used to demonstrate altered levels of 15 blood proteins, and one of the proteins was TSH (Mizejewski et al., 2013). Reduced levels of TSH were observed in bloodspots from infants who later were found to have ASD (n=16 ASD and n=32 TD; gender not reported). These data indicate that the reduced TSH was present at birth. More recently, maternal mid-pregnancy serum TSH levels were found to be inversely correlated with the likelihood of having a child with ASD (Yau et al., 2015). This study used 149 control children and 78 ASD children, and both genders were in the two groups.

When ADOS subdomain scores were compared with TSH levels, there was a significant negative correlation with Social Interaction, Communication+Social interaction, and SBRI such that a higher subdomain score (i.e., more ASD symptoms) was correlated with lower TSH levels. These data suggest that TSH may not only serve as an important member of an ASD biomarker panel, but it may also represent a useful index of an ASD phenotype.

Proinflammatory cytokines (PICs) have been reported to be elevated in ASD children (e.g., Ashwood et al., 2011). In the present study using both the RBM and MSD platforms, the inventors found 17-30% increases in IL-8 in the serum of ASD boys. In the study of (Mizejewski et al., 2013)], elevations in IL-8 were reported in ASD children in bloodspots collected at birth. PICs are directly linked with neuroinflammation. Elevations in plasma IL-8 (33%) have also been reported by Suzuki et al. (Suzuki et al., 2011) in high-functioning ASD boys, with a mean age of 12 years (n=28 ASD boys and n=28 TD boys). Finally, in a meta-analysis of three studies, Masi et al. [31] report significant elevations in IL-8 in 150 ASD vs. 140 TD children (primarily boys). There was no significant relationship between ADOS and IL-8 scores or between protein levels and ASD sub-groups (Non-Verbal, GI concerns, and Regression). These data suggest that IL8 levels are not specific to the subdomains used to diagnose ASD.

There are a number of limitations in this study. The relatively small sample size renders the data presented here preliminary, and a larger study with more ASD subjects is underway. The increased prevalence of ASD in boys resulted in the study primarily focusing on boys, which does not allow one to thoroughly investigate gender-specific differences. Examination of TSH and IL-8 in ASD and TD girls should be further evaluated in a larger study. When making electro-chemiluminescent measurements on 96-well plates, there are often plate-to-plate differences that add variability to the data, however, the inventors routinely run standards on every plate as well as calibration curves to minimize this source of data variability.

In order to identify ASD at an early age, and facilitate treatment before symptoms manifest, robust biomarkers are important. The levels of TSH were significantly lower in ASD boys vs. TD boys, and the levels were highly correlated with ADOS subdomain scores, suggesting that TSH levels may be useful for assessing specific ASD phenotypes. It is also interesting that when the inventors analyzed the accuracy of predicting ASD vs. TD using more than one analyte, they found that the accuracy went from 74-76% for single analytes, to 82% when using TSH and IL-8 together. The use of panels of blood proteins for disease identification and/or characterization appears to be a useful strategy, and one that the inventors will pursue by (i) testing a larger validation set of ASD and TD samples on the MSD platform, and (ii) looking at a total of four analytes previously identified in the RBM platform (e.g., apolipoprotein E and stem cell factor along with TSH and IL-8) to determine whether four protein analytes combined will provide an accuracy of −90% in predicting ASD in boys.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
Amaral, Brain Research, 1380:3-9, 2011.
American Psychiatric Association. Desk Reference to the Diagnostic Criteria from DSM-5. 5th ed. Washington, D.C.: American Psychiatric Association (2013).
Anderson et al., Thyroid, 13:1039-1056, 2003.
Ashwood et al, Brain Behay. Immuni., 25(1):40-45, 2011.
Ashwood et al., Brain, Behay. Immun., 25:840-849, 2011.
Ashwood, et al., J. Leukocyte Biol, 80:1-15, 2006.
Bernal, Vitamins & Hormones, 71:95-122, 2005.
Breiman, Machine Learning, 45(1):5-32, 2001.
Burguillos, Meth. Mol. Biol. 1041:93-100, 2013.
Curran et al., Psychol Meth, 1:16-29, 1996.
De Jager et al., Semin. Nucl. Med., 23(2):165-179, 1993.
Doolittle and Ben-Zeev, Methods Mol Biol, 109:215-237, 1999.
Enstrom et al., Brain Behav Immun 23:124-133, 2009.
Enstrom et al., Curr Opin Investig Drugs, 10:463-473, 2009.
Fiandaca et al., Alz. & Dement, 1-8. aug 14. pii: s1552-5260(14)02469-8. doi: 10.1016/j.jalz.2014.06.008. [epub ahead of print] (2014).
Figliozzi et al., Methods in Enzymology, 267:437-447, 1996.
Glatt et al. (2012) J. American Acad. Childhood and Adolescent Psychiatry. 2012 September; 51(9):934-44.e2. doi: 10.1016/j.jaac.2012.07.007. Epub 2012 Aug. 2.
Graham, Missing Data: Analysis and Design., New York, N.Y.: Springer, 2012.
Gubbels, Cell Immunol. February 10. pii: S0008-8749(15)00029-5. doi: 10.1016/j.cellimm.2015.02.002. [Epub ahead of print] (2015).
Gulbis and Galand, Hum. Pathol., 24(12):1271-1285, 1993.
Hahn & Tsao, In: Dubois' Lupus Erythematosus, 4^{th} Ed., Wallace & Hahn (Eds.), Lea and Febiger, Philadelphia, 195-201, 1993.
Hashimoto et al., Dev. Med. Child. Neurol. 33(4):313-319, 1991.
Heuer et al., Autism Res., 1(5):275-283, 2008.
Hewitson, O A, Autism, 1(1):1-7, 2013.
Jyonouchi et al., Neuropsychobiol 51, 77-85, 2005.
Jyonouchi et al., Neuropsychobiology, 46:76-84, 2002.
Koibuchi and Iwasaki, Endocrinol. J. 53:295-303, 2006.
Mapstone et al., Nature Med., 20:415-418, 2014.
Mizejewski et al., Biomarkers in Medicine, 7.2:247, 2013.
Molloy et al., J Neuroimmunol, 172:198-205, 2006.
Momeni et al., (2012). Translational Psychiatry. 2012 Mar. 13; 2:e91. doi: 10.1038/tp.2012.19.
Morreale et al., Eur. J. Endocrinol. 151 Suppl 3:U25-37, 2004.
Nakamura et al., In: Handbook of Experimental Immunology (4th Ed.), Weir et al. (Eds), 1:27, Blackwell Scientific Publ., Oxford, 1987.
Napolioni et al., J. Neuroinflamm. 10:38-48, 2013.
Ngounou Wetie et al. (2015). Autism Research January 27. doi: 10.1002/aur.1450. [Epub ahead of print]
O'Bryant et al., J. Alzheimers Dis., 42:1325-1335, 2014.
Olivos et al., Org. Lett., 4:4057-4059, 2002.
O'Roak et al., Nature Communications, 5, Article number:5595doi:10.1038/ncomms6595, 2014.
Pardo et al., Int Rev Psychiatry, 17:485-495, 2005.
Pramparo et al., JAMA Psychiatry, DOI:http://dx.doi.org/10.1001/jamapsychiatry, 2015.
Quan et al., Journal of Immunological Methods, 402:23-34, 2014.
Reddy et al., Cell, 144:132-142, 2011.
Schaafsma & Pfaff (2014). Frontiers in Neuroendocrinology, 35(3): 255-271.
Schwarz et al., Mol. Psychiat., 16(12):1213-1220, 2011.
Singh, Ann Clin Psychiat, 21:148-160, 2009.
Spratt et. al., Int. Neuropsychiat. Dis. J. 3(1):19-26, 2014.
Steeb et al., Molecular Autism. January 27; 5(1):4. doi: 10.1186/2040-2392-5-4, 2014.
Suzuki et al., PLoS One 6(5):e20470, 2011.
Villeda et al., Nat Med., 20(6):659-663, 2014.
Warren, et al., Immuno Invest, 19:245-251, 1990.
West et al, PLoS ONE, 9(11):e112445, 2014.
Wills et al., Brain Behav Immun, 23:64-74, 2009.
Yau et al., J. Autism Develop. Disord. 45(3):719-730, 2015.
Yuen et al., Nature Medicine, published online 26 Jan. 2015; doi:10.1038/nm.3792, 2014.

1. A method of identifying a male subject having or at risk of developing Autism Spectrum Disorder (ASD) comprising:
(a) providing a blood product sample from a male subject;
(b) determining the levels of thyroid stimulating hormone (TSH) and interleukin 8 (IL-8) said sample; and
(c) identifying said subject as having or at risk of developing ASD when the level of IL-8 are elevated as compared to levels observed in normal subjects, when the level of TSH are reduced as compared to levels in normal subjects.

2. The method of claim 1, further comprising determining the level of antibodies binding to a peptoid having the following structure: 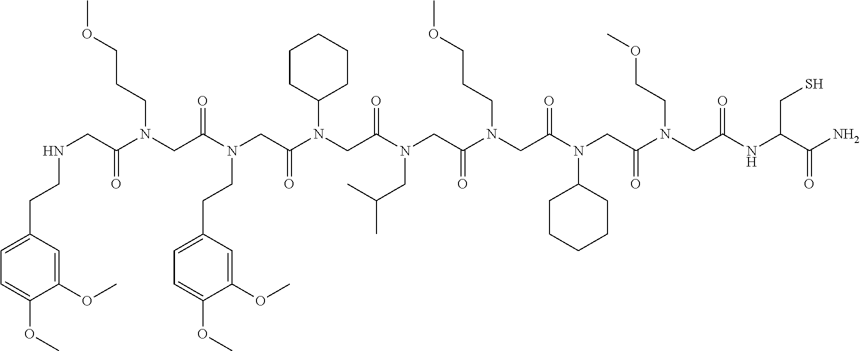 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]
and further identifying said subject as having or at risk of developing ASD when the level of said antibodies is reduced as compared to levels observed in normal subjects.

3. The method of claim 1, wherein said subject is suspected as having ASD.

4. The method of claim 1, wherein said sample is whole blood or serum.

5. The method of claim 1, wherein said subject is age about 12 or younger.

6. The method of claim 1, wherein said subject is about 1-10 years of age.

7. The method of claim 1, wherein said subject is about 2-8 years of age.

8. The method of claim 1, wherein said subject is about 1 year of age.

9. The method of claim 1, wherein said peptoid is located on a solid support, and determining in step (c) comprises measuring antibodies bound to said solid support.

10. The method of claim 9, wherein said solid support is a bead, a chip, a filter, a dipstick, a slide, a membrane, a polymer matrix, a plate or a well.

11. The method of claim 1, wherein determining in step (b) comprises a quantitative immunoassay.

12. The method of claim 11, wherein said quantitative immunoassay comprises ELISA, RIA, FIA, and electrochemiluminescence.

13. The method of claim 1, further comprising obtaining the sample from the subject.

14. The method of claim 1, further comprising examining the level of one or more of ferritin, alpha 1 microglobulin, apolipoprotein E, apolipoprotein H, AXL receptor tyrosine kinase, chromogranin A, monocyte induced by gamma interferon, monocyte chemotactic protein 4, and/or stem cell factor in said sample.

15. The method of claim 1, wherein the antibodies of step (c) are IgG1 antibodies.

16. A peptoid having the formula:

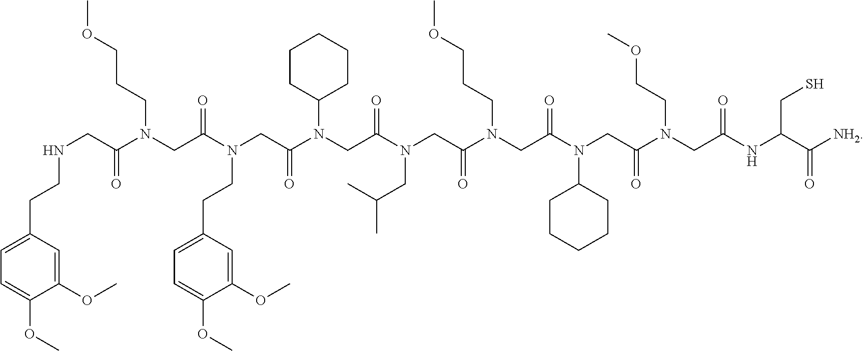 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

17. A solid support comprising fixed thereto a peptoid having the formula:

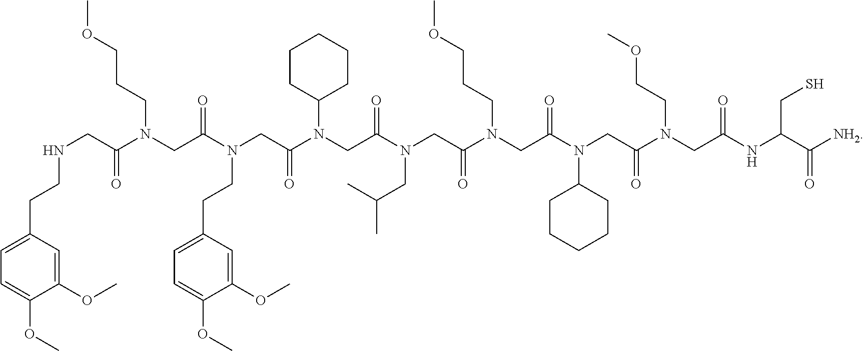 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

18. The solid support of claim 17, wherein said solid support is a bead, a chip, a filter, a dipstick, a slide, a membrane, a polymer matrix, a plate or a well.

19. The solid support of claim 17, further comprising one or more agents for performing a positive-control and/or negative-control reactions for analytes found in a blood sample.

20. A kit comprising a peptoid having the formula:

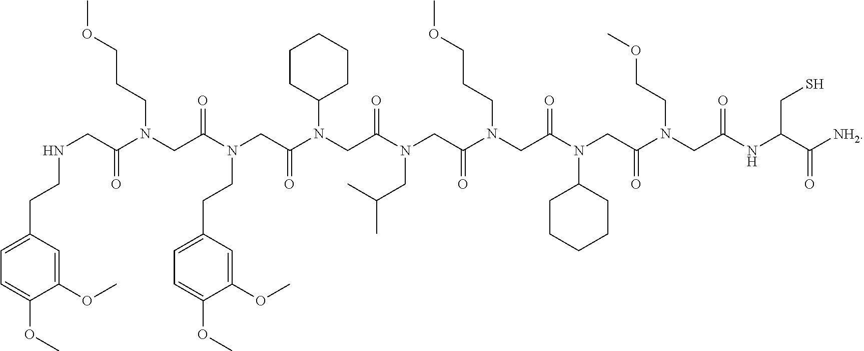 [SMILES: COCCCN(CC(=O)N(CCc1ccc(OC)c(OC)c1)CC(=O)N(CC(=O)N(CC(=O)N(CCCOC)CC(=O)N(CC(=O)N(CCOC)CC(=O)NC(CS)C(N)=O)C1CCCCC1)CC(C)C)C1CCCCC1)C(=O)CNCCc1ccc(OC)c(OC)c1]

21-25. (canceled)
